(19) United States
(12) Patent Application Publication
(10) Pub. No.: US 2009/0226493 A1
(43) Pub. Date: Sep. 10, 2009

(54) Agglomerates of Sucralose and Polyols, and their Use in Chewing Gums

(75) Inventors: Michel Lopez, Nogent-sur-Seine (FR); Warren L. Nehmer, Decatur, IL (US)

Correspondence Address: Ratner Prestia, P.O. Box 980, Valley Forge, PA, 19482, US

(73) Assignee: Tate & Lyle Technology Ltd., London (GB)

(21) Appl. No.: 12/394,530

(22) Filed: Feb. 27, 2009

Related U.S. Application Data
(60) Provisional application No. 61/067,675, filed on Feb. 29, 2008.

(51) Int. Cl. A61K 8/02; A23G 4/00; A23G 4/18; A61K 9/68

(52) U.S. Cl. 424/401; 426/3; 426/5; 424/440

(57) ABSTRACT

The invention provides co-agglomerates of sucralose and a polyol, wherein the co-agglomerate comprises grains of polyol onto which sucralose has been dried, as well as methods for making such co-agglomerates. In general, the methods comprise contacting a dry powder of polyol with a liquid solution of sucralose in a food acceptable solvent under conditions which moisten, but not dissolve the polyol powder, to form a moist mixture, and drying the moist mixture to form a co-agglomerate comprising grains of agglomerated polyol onto which sucralose has been dried. Also provided are artificial sweetener carriers comprising bentonite which comprise sucralose. The invention features pharmaceutical, cosmetic, and food compositions, especially chewing gum, comprising a co-agglomerate of the invention, comprising a bentonite carrier, or comprising both. The invention further features methods of enhancing the sweetness perception during the onset phase of chewing, a method of enhancing the sweetness build-up in sweetness perception and a method of enhancing the maximum sweetness perception of food, pharmaceutical or cosmetic compositions.

### CROSS REFERENCE TO RELATED APPLICATIONS

This application claims priority to U.S. Provisional Application Ser. No. 61/067,675, filed Feb. 29, 2008, the entire contents of which are incorporated by reference herein.

### FIELD

This invention relates to a co-agglomerate of sucralose and a polyol for food, pharmaceutical or cosmetic compositions, in particular chewing gum containing sucralose as sweetener which modifies the sweetness profile of sucralose. Further, the invention relates to methods of enhancing the sweetness perception using the compositions of the invention and to a method of producing the co-agglomerates of the invention.

### BACKGROUND

Chewing gums comprise many ingredients including sweeteners. Sweeteners suitable for chewing gums include both natural and artificial sweeteners. Recently, the high intensity artificial sweeteners have been the focus of investigation for use in chewing gum.

High intensity sweeteners may be hundreds of times sweeter than natural sweeteners such as sucrose and glucose. High intensity sweeteners of recent interest include aspartame, acesulfame K, cyclamates, saccharin, and sucralose.

Sucralose is a new high intensity sweetener which is a tri-chlorinated sucrose derivative and is about 600 times sweeter than sucrose. Sucralose has been reported to be much cheaper than aspartame and at least 3 times sweeter. Sucralose has a sweet taste similar to sucrose. Chemically, sucralose is known as 4,1′,6′-trichloro-4,1′,6-trideoxy-galactosucrose, or alternatively 1,6′-dichloro-1,6-dideoxy-(3)-D-fructofuranosyl 4-chloro-4-deoxy-(a)-D-galactopyranoside. In addition, sucralose is also referred to in short from as TGS. Its use to sweeten substances including oral compositions is disclosed in U.S. Pat. Nos. 4,343,934 and 4,389,394 which are incorporated herein by their reference.

Depending on the application, different sweetness profiles are desirable. The sweetness profile of a food composition includes an onset phase (approximately 10 seconds beginning with intake in the person's mouth), the build-up of sweetness (the rate at which the sweetness is built up to a maximum), the maximum sweetness which may form a plateau, and the phase of sweetness lingering in which the sweetness perception gradually dies out.

To get the desired sweetness profile in low calorie or sugar free food, different high intensity sweeteners (HIS) of different profiles can be combined. The overall sweetness profile of a composition of high intensity sweeteners (HIS combo) depends on the sweetness profile of each sweetener and on the food matrices. Typically, the overall sweetness profile is the predictable result of a more or less straightforward addition of sweetness profiles of the individual sweetener ingredients.

A typical conventional HIS combo is a formulation of aspartame or sucralose with acesulfame-K, which formulation finds usage e.g., in beverages, dairy, baking and confectionery. The acesulfame-K induces the perception of a faster sweetness onset, whilst the aspartame or sucralose components are responsible of the sweetness lingering. Another objective in food is to extend the flavour perception. Davidson et al. described in 1999 that the loss of sweetness (sucrose) from a chewing gum caused a large loss in the perceived mintiness while menthone flavour is still released (Davidson et al., J. Agric. Food Chem. 1999, 47, 4336).

WO 88-08672 A1 addresses the problem of providing a chewing gum having improved sweetness and flavour extension in combination with an initial sweetness impact upon chewing. The document proposes the combination of a chewing gum composition comprising a mixture of chewing gum ingredients comprising at least a chewable gum base and a sweetener comprising an effective amount of sucralose and a fast release sweetener. The authors provide examples of sugar-free chewing gums wherein sucralose is added as powder or solution in an organic solvent to the remaining ingredients of the chewing gum. The authors merely speculate that the rate of release of sucralose may further be controlled, for example, by dissolution in a food acceptable organic solvent, encapsulating the sucralose with a further ingredient or co-drying the sucralose with a further ingredient. The authors speculate that such treatment may lead to an increase in initial release of sweetness and flavour and respective experience by the consumer. The document, focussing on additively balancing the release profile by the combination of sweeteners of differing release characteristics, provides no examples for sucralose containing sweetener obtained by co-drying or encapsulating treatment. The document is furthermore silent on any particular compound, sucralose is to be combined with by co-drying or encapsulation.

Co-drying sucralose involves evaporating a solvent from a solution of sucralose and at least one further ingredient. A widespread method of producing co-dried granules is spray-drying. The method results in particles consisting of an intimate mixture of the ingredients on a molecular level. The co-drying or spray-drying method provides the formation of a homogeneous distribution of the ingredients in the granules.

In the art therefore there still remains the problem of enhancing the sweetness perception during the onset phase of chewing a sugar free food, pharmaceutical or cosmetic composition by incorporation of sucralose as opposed to adding undesirable conventional fast release sweeteners.

In particular, it is desirable to provide an alternative to the combo aspartame/acesulfame-K (Asm/AcK, sometimes referred to as Apm/AcK) e.g., in chewing gum matrices in order to avoid required labelling on the package of warnings of a source of phenylalanine.

### SUMMARY

The invention provides a co-agglomerate of sucralose and a polyol, wherein the co-agglomerate comprises grains of polyol onto which sucralose has been dried.

With the co-agglomerate of the invention, food compositions, pharmaceutical compositions and cosmetic compositions become available, in which the conventional polyol powders and/or the HIS composition (HIS combo) has been totally or partially been replaced by the co-agglomerate of the invention. The percentage of co-agglomerate of sucralose and polyol varies depending on the ratio sucralose to polyol and the final sucralose content of the product composition.

Food compositions, pharmaceutical compositions and cosmetic compositions using the co-agglomerate of the invention show enhanced sweetness perception during the onset phase of chewing and increased maximum sweetness as compared to comparable compositions having identical ingredients, wherein sucralose and the polyol has been added separately. Thus, the invention provides the option to reduce the amount of HIS combo without sacrificing maximum sweetness.

The food compositions, pharmaceutical compositions, and cosmetic compositions can further comprise a bentonite carrier comprising sucralose. The sucralose can be absorbed into, encapsulated in, and/or granulated on top of the bentonite. The sucralose-containing bentonite carrier can further comprise a hydrocolloid coating. Nonlimiting examples include a guar gum coating, an iota carrageenan coating, or a gum arabic coating.

Chewing gum with enhanced and sustained sweetness is a preferred feature of the invention. The chewing gum can comprise a component for rapid flavor release and a component for extended flavor release. The first component can comprise a co-agglomerate comprising grains of polyol onto which sucralose has been dried, and the second component can comprise bentonite into which sucralose has been absorbed, and onto which sucralose has been granulated. The second component can further comprise a hydrocolloid coating, for example, a gum arabic coating.

In another aspect, the invention provides a method of enhancing the sweetness and/or the flavour perception by an individual during the onset phase of chewing or eating a food composition or a pharmaceutical composition or a cosmetic composition, wherein the method comprises:

providing a food composition or a pharmaceutical composition or a cosmetic composition according to the invention; taking the food composition into the individual's mouth; and starting chewing and/or eating the food composition.

In a further aspect of the invention, there is provided a method of enhancing the sweetness build-up in sweetness perception and/or the maximum sweetness perception by an individual during chewing or eating a food composition or a pharmaceutical composition or a cosmetic composition, wherein the method comprises:

providing a food composition or a pharmaceutical composition or a cosmetic composition according to the invention; taking the food composition into the individual's mouth; and, starting chewing and/or eating the food composition.

In still another aspect of the invention, there is provided a method of making the co-agglomerate of the invention, the method comprising the steps of contacting a dry powder of polyol with a liquid solution of sucralose in a suitable solvent under conditions which moisten, but not dissolve the polyol powder, to form a moist mixture, and drying the moist mixture to form a co-agglomerate comprising grains of agglomerated polyol onto which sucralose has been dried. The polyol powder can be pretreated, including by agglomeration or extrusion.

### BRIEF DESCRIPTION OF THE DRAWINGS

FIG. 1 shows the average sweetness perception time-intensity curves for the three gum samples examined in example 4. Each curve is generated from a total of 40 individual curves (ten panelists, four replicates). The y-axis is sweetness intensity (arbitrary units 0-100) and the x-axis is time (seconds 0-600).

FIG. 2 shows the average mintiness perception time-intensity curves for the three gum samples examined in example 4. Each curve is generated from a total of 40 individual curves (ten panelists, four replicates). The y-axis is sweetness intensity (arbitrary units 0-100) and the x-axis is time (seconds 0-600).

FIG. 3 shows the sweetness profile of various gum formulations over time.

FIG. 4 shows the particle size distribution of experimental sucralose samples.

FIG. 5 shows the sweetness profile of various coated and uncoated gum formulations over time.

FIG. 6 shows the effect of hydrocolloid coating and sorbitol/sucralose agglomerate on the relative sweetness of chewing gum at each minute of chewing. One minute, FIG. 6A; Two minutes, FIG. 6B; Three minutes, FIG. 6C; Four minutes, FIG. 6D; Five minutes, FIG. 6E.

FIG. 7 shows the combined sweetness profile for coated bentonite/sucralose, uncoated bentonite/sucralose and the sorbitol/sucralose agglomerate.

FIG. 8 shows the particle size distribution of granulated bentonite with sucralose utilized in Example 7.

FIG. 9 shows the sweetness profile for granulated bentonite with sucralose in chewing gum.

FIG. 10 shows the sweetness profile for sorbitol/sucralose agglomerate in chewing gum.

FIG. 11 shows the particle size distribution of experimental sucralose samples utilized in Example 8.

FIG. 12 shows the sweetness profile for chewing gum samples utilized in Example 8.

FIG. 13 shows the maximum sweetness of chewing gum at 1000 and 1500 ppm sucralose.

FIG. 14 shows the effect of sucralose level in chewing gum on its relative sweetness release.

FIG. 15 shows the effect of sucralose level in chewing gum on its maximum sweetness.

FIG. 16 shows the effect of sucralose form in chewing gum on its relative sweetness release.

FIG. 17 shows the effect of sucralose form in chewing gum on its maximum sweetness.

FIG. 18 shows the effect of sucralose encapsulation level on its relative sweetness release in chewing gum.

FIG. 19 shows the effect of sucralose encapsulation level on its maximum sweetness in chewing gum.

### DETAILED DESCRIPTION

The co-agglomerates of the invention are different from granules obtained by co-drying/spray-drying or encapsulating sucralose. It is to be noted that the products obtained by spray-drying are sometimes called “agglomerates”, and the products obtained by co-spray-drying are sometimes called “co-agglomerates” in the art. However, those products obtained from co-spray-drying are fundamentally different from the co-agglomerates according to the invention. While co-drying/spray-drying of sucralose with a further ingredient results in granules or agglomerates having a distribution of ingredients in an intimate mixture on a molecular level, the term “co-agglomerate”, within the context of the present invention, defines co-agglomerates which comprise grains of agglomerated polyol and sucralose which is isolated from the polyol on a molecular level. Therefore the term “co-agglomerate” as used herein excludes intimately mixed products such as those obtained by co-drying/spray-drying.

Preferably, the sucralose forms an at least partial coating or particles on the grains of agglomerated polyol. The sucralose may also form a full coating on a grain of polyol, though typically the sucralose will form particles on the polyol, not forming a complete shell on the polyol grains. The polyol is best use as a dry polyol powder, wherein the polyol grains may be of any form and size, as long as the polyol does not form an intimate mixture with sucralose. The size of the polyol grains preferably varies in the range of from 53 up to 700 μm and more preferably 53 to 500 μm. But larger grains will not pose any problems for the invention. Essentially, the grain size will vary depending on the physical parameters desired in the final product.

As is shown by the examples, such co-agglomerate according to the invention results in a significantly increased sweetness onset perception as compared to conventional sorbitol/sucralose with both components added separately or both components added as a co-spray dried mixture. However, the significant increase in sweetness onset perception is not correlated with an equally increased sucralose release in the mouth of the consumer. In contrast, the effective measured release of sucralose was largely unaffected by the particular form in which sucralose was added to the mixture of chewing gum components (added separately or as a co-agglomerate with sorbitol).

Furthermore, the comparative experiments reproduced below have shown that a composition according to the invention comprising a co-agglomerate of sucralose and a polyol exhibits an enhanced sweetness build-up and an increased maximum sweetness as perceived by the consumer in comparison to a composition having both components added separately.

It was found that a sample containing spray-dried sorbitol-sucralose mixture was not significantly different in sweetness onset from a sugar-free chewing gum (SFG) containing sucralose and sorbitol added separately, but showed slower sweetness-onset than the sample according to the invention, containing a co-agglomerated sorbitol/sucralose combo.

A sample according to the invention, containing a co-agglomerated sorbitol/sucralose combo, in contrast had a faster sweetness onset perception by the consumer than SFG containing sucralose added separately. The co-agglomerated sample showed furthermore a significantly higher maximum sweetness level.

The co-agglomerate of sucralose with a polyol offers many advantages for food compositions according to the invention, such as:

it is an alternative to the combo acesulfame-K/aspartame in chewing gum matrices;

as a technological approach, the agglomeration replaces the combo of two HIS by avoiding the use of acesulfame-K in chewing gum matrices;

it allows to simplify the labelling on the SFG packaging,

The polyol used according to the invention may be any C4 to C12 polyol. Preferably, the polyol is a sugar alcohol, most preferably the polyol is selected from the group consisting of erythritol, xylitol, sorbitol, mannitol, maltitol, isomalt, lactitol, and mixtures thereof. In particular, the polyol is sorbitol and/or maltitol.

The co-agglomerate according to the invention is preferably characterized by a sucralose-polyol ratio in the range of 0.05 wt.-% to 40 wt, wherein the polyol is preferably sorbitol.

Particularly preferred is a sucralose-polyol ratio in the range of 0.15 wt.-% to 2.20 wt.-%, more preferably in the range of 0.20 wt.-% to 0.70 wt.-%, even more preferably in the range of 0.25 wt.-% to 0.40 wt.-%, wherein the polyol is preferably sorbitol. Comparative experiments reproduced below have surprisingly shown that an optimum balance in sweetness onset, sweetness build up, maximum sweetness and sweetness lingering time as perceived by the consumer may be achieved within these concentration ratios.

In a further particularly preferred embodiment, the co-agglomerate has a sucralose-polyol ratio in the range of 5 wt.-% to 40 wt.-%, preferably 10 wt.-% to 25 wt.-%, wherein the polyol is preferably sorbitol. Further to the surprising enhancement of sweetness onset perception by the consumer as compared to the polyol and the sucralose added separately, within these ratios the additional advantage is provided that the manufacture of the food composition, such as a sugar-free chewing gum, is much more flexible and easier to handle, because the producer of e.g., sugar-free chewing gum (SFG) must add less than 1% in the cp when the recipe contains 0.1% sucralose and the ratio of sucralose in the co-agglomerate is higher than 10%. It is a major advantage for food producers to use co-agglomerate with maximum sucralose/polyol ratio to decrease the quantity of such new ingredient in the final formulation and therefore limit or avoid any change in the cp texture, with respect to e.g., hardness or flexibility of chewing gum.

In a further embodiment, the invention relates to a method of making a co-agglomerate according to the invention, the method comprising the steps:

contacting a dry powder of polyol with a liquid solution of sucralose in a food acceptable solvent under conditions which moisten, but not dissolve the polyol powder, to form a moist mixture;
drying the moist mixture to form a co-agglomerate comprising grains of agglomerated polyol onto which sucralose has been dried;
adding the co-agglomerate to a food composition.

The sucralose solution may have any suitable sucralose concentration. However, preferred concentrations range from 1 g/l to saturation, more preferred from 10 g/l to 250 g/l. The polyol powder used in the process of the invention may have various degrees of porosity.

The invention also relates to a food composition obtained or obtainable by a method of the invention as described above.

Food, pharmaceutical or cosmetic compositions may comprise a co-agglomerate according to the invention in practically any desired amount. Typically, the co-agglomerate is contained in an amount in the range of 10 to 90 wt.-%, preferably 30 to 70 wt. %, best, e.g., in the case of a SFG, in the range of from 40 to 60 wt.-%.

The invention also relates to carriers for artificial sweeteners, including sucralose. In some aspects, the carriers may be used to extend the release of the sweetener, including the co-agglomerates described and exemplified herein, in various food, cosmetic, or pharmaceutical compositions.

The carriers preferably comprise silica or clay carrier agents such as bentonite, kaolin, laponite, or mixtures thereof. Bentonite is particularly preferred. Thus, for example, bentonite carriers can comprise sucralose. The sucralose is preferably absorbed into the bentonite, but can also be encapsulated within the bentonite. In some aspects, the sucralose can also be granulated on top of the bentonite. The sucralose can be part of a co-agglomerate with a polyol. Carriers comprising sucralose can further comprise a hydrocolloid coating. Suitable hydrocolloid coatings include, but are not limited to alginate, agar, carrageenan, iota carrageenan, chitin, chitosan, cyclodextrin, galactomannan, gellan, guar gum, gum arabic, insulin, laminarin, locust bean, maltodextrin, pectin, tara, tragacanth, and xanthan. Gum arabic is particularly preferred.

Preferred compositions according to the invention are chewing gums, in particular sugar-free chewing gum (SFG).

In general, a chewing gum composition comprises a water soluble bulk portion and a water insoluble chewable gum base portion and, typically water insoluble flavouring agents. The water soluble portion dissipates with a portion of the flavoring agent over a period of time during chewing. The gum base portion is retained in the mouth throughout the chew.

The insoluble gum base generally comprises elastomers, resins, fats and oils, waxes, softeners and inorganic fillers. Elastomers may include polyisobutylene, isobutylene-isoprene copolymer, styrene butadiene rubber as well as natural latexes. Resins include polyvinylacetate and terpene resins.

Fats and oils may also be included in the gum base, including tallow, hydrogenated and partially hydrogenated vegetable oils, and cocoa butter. Commonly employed waxes include paraffin, microcrystalline and natural waxes such as beeswax and carnuba. According to the present invention, the insoluble gum base constitutes between about 5 to 95 weight percent of the gum. Preferably the insoluble gum base comprises about 10 to about 50 weight percent of the gum and more preferably about 25 to about 35 weight percent.

The gum-base typically also includes a filler component. The filler component such as calcium carbonate, magnesium carbonate, talc, dicalcium phosphate and the like. The filler may constitute between about 5 to about 60 weight percent of the gum base. Preferably, the filler comprises about 5 to 50 weight percent of the chewing gum base.

Gum bases typically also contain softeners, including glycerol monostearate and glycerol triacetate. Further, gum bases may also contain optional ingredients such as antioxidants, colours, and emulsifiers. The present invention contemplates employing any commercially acceptable gum base.

The water soluble portion of chewing gum conventionally generally comprises a sweet, powder bulking agent which is most often a sugar each as sucrose, dextrose, maltose or isomaltulose; or a sugar alcohol such as sorbitol, mannitol, isomalt, maltitol, or xylitol. Mixtures of two or more of these bulking agents are conventionally used in chewing gums.

The water soluble portion of chewing gum may further comprise softeners, sweeteners, flavoring agents and combinations thereof. Softeners are added to the chewing gum in order to optimize the chewability and mouth feel of the gum. Softeners, also known in the art as plasticizers or plasticizing agents, generally constitute between about 0.5 to about 15.0 weight percent of the chewing gum. Softeners contemplated by the present invention include glycerine, lecithin, and combinations thereof. Further, aqueous sweetener solutions such as those containing sorbitol, hydrogenated starch hydrolysates, corn syrup and combinations thereof may be used as softeners and binding agents in gum.

Therefore, the invention also provides a chewing gum comprising one or more or a mixture of chewing gum ingredients selected from the group consisting of chewable gum base, flavor ingredient, bulking agent ingredient, softener ingredient, as defined above.

In a particularly preferred embodiment of the invention, the flavor ingredient comprises mint, though alternatively or in addition, the food, pharmaceutical or cosmetic composition according to the invention may contain other natural or artificial flavors, such as fruit (e.g., apple, banana, cherry, orange, pear, pineapple), herb (cinnamon, wintergreen, vanilla) nuts (bitter almond, hazelnut), or chocolate or coffee as a non-limiting selection of possible flavors. The applicants have shown that the compositions according to the invention, comprising a co-agglomerate of sucralose and a polyol provide for a higher intensity of the perceived sweet and flavor during chewing.

In addition to, or in lieu of, the sucralose and polyol co-agglomerates described and exemplified herein, the food compositions can comprise one or more artificial sweetener carriers, preferably for use in extending the release of the sweetener to prolong flavor and/or sweetness perception during consumption. The carriers preferably comprise an artificial sweetener. Bentonite is a preferred carrier.

Bentonite carriers can comprise sucralose. The sucralose can be absorbed into or otherwise interspersed with the bentonite, and can also be encapsulated within the bentonite. In some aspects, the sucralose can be granulated on top of the bentonite, can be absorbed into the bentonite, and can be absorbed into and granulated on top of the bentonite. The sucralose can be part of a co-agglomerate with a polyol.

Carriers, including bentonite, comprising sucralose can further comprise a hydrocolloid coating. Suitable hydrocolloid coatings include, but are not limited to guar gum, iota carrageenan, and gum arabic. Gum arabic is particularly preferred.

In some embodiments of the invention, the carrier comprising sucralose may be provided in admixture with a co-agglomerate as described above. The carrier comprising sucralose and the co-agglomerate may be provided in any ratio, depending on the desired effect. In some embodiments, the ratio may be at least 10:90, or at least 30:70, or at least 45:55. In some embodiments, the ratio may be at most 90:10, or at most 70:30, or at most 55:45. Such mixtures may be provided alone or in a mixture with other ingredients. In some embodiments, the other ingredients are those for making a chewing gum, thereby providing a gum having both extended sweetness release and enhanced sweetness during the onset phase of chewing.

Chewing gums are preferred food compositions comprising artificial sweetener carriers. Particularly preferred chewing gum compositions comprise both a sucralose-polyol co-agglomerate and a sweetener carrier comprising sucralose. Such chewing gum thus can be characterized as having a component for rapid sweetness release, the co-agglomerate, and a component for extended sweetness release, the carrier.

From the foregoing it becomes apparent that the invention also provides a method of enhancing the sweetness perception by an individual during the onset phase of chewing or eating a food composition or a pharmaceutical composition or a cosmetic composition, wherein the method comprises:

providing a food, pharmaceutical, or cosmetic composition according to the invention;
taking the composition into the individual's mouth;
starting chewing and/or eating the composition.

Furthermore, the invention provides a method of enhancing the flavour perception by an individual during the onset phase the maximum and the lingering phase of chewing or eating a food composition wherein the method comprises:

providing a food, cosmetical or cosmetic composition according to the invention;
taking the composition into the individual's mouth;
starting chewing and/or eating the composition. Enhancing mintiness perception is most preferred.

Furthermore, the invention provides a method of enhancing the sweetness build-up in sweetness perception by an individual during the onset phase of chewing or eating a food composition wherein the method comprises:

providing a food, cosmetical or cosmetic composition according to the invention;
taking the composition into the individual's mouth;
starting chewing and/or eating the composition.

Furthermore, the invention provides a method of enhancing the maximum sweetness perception by an individual during chewing or eating a food composition wherein the method comprises:

providing a food, cosmetical or cosmetic composition according to the invention;
taking the composition into the individual's mouth;
starting chewing and/or eating the composition.

Without wanting to be bound by theory, the inventors assume that an interaction between HIS and solid bulking agents modifies the sweetness profile of sweeteners such as sucralose either inducing a faster sweetness onset, higher maximal sweetness or sustained sweetness lingering.

This is probably related to the physical distribution of the sucralose in the combo, either as a superficial co-agglomeration on a polyol grain (such as an at least partial coating) or as a thoroughly mixed component (spray-dried). The intimate association between sucralose-sorbitol in a spray-dried combo is the necessary result of co-dissolution of both components prior to atomisation in a spray tower while on the co-agglomeration approach according to the invention a solution of sucralose is sprayed on sorbitol powder prior to drying. Such spraying is carried out in principle from any direction, such as preferably from the top (top-spraying) or from the bottom of a fluidized bed of polyol powder (bottom-spraying).

The following examples are illustrative of the invention but are not to be regarded as limiting.

In this application sugar free chewing gum (SFG) has been chosen as model wherein the major bulking agents are dry polyols such as sorbitol and maltitol. Various intimate combos between sucralose and dry sorbitol have been prepared using spray drying and co-agglomerating technologies. Moreover, trained panelists have ranked the sweetness perception during the three phases of a chewing gum chew: initial chew (10 s) where the gum becomes hydrated, intermediate chew where flavour and sweetness are released and final chew when only the gum remains. The ranking sweetness criteria were the sweetness onset time is the rate at which the first signs of sweetness appear, the sweetness build-up and the maximal sweetness level.

### Example 1

### Co-Agglomeration Vs. Separately Adding or Co-Spraying

1.1 The experiment deals with the effect of the way sucralose is added on the sweetness perception profile of chewing gum sticks. Four sugar free sticks have been produced, containing sorbitol & sucralose added separately, intimately associated by sorbitol/sucralose spray-dried, or sorbitol/sucralose agglomerated. The fourth stick did not contain any sucralose.

All these sticks have been tasted and compared by a sensory panel to check possible differences in sweetness onset time.

Co-agglomerated samples 4, 6, 7 and 8 were produced by top-spraying an aqueous solution of sucralose onto a fluid-bed of sorbitol powder in a Glatt GPCG5 pilot plant batch reactor. The reactor was loaded with 3 kg sorbitol powder. Top-spraying is a widely used technology for making (partial) coatings. According to the following protocol the reactor was loaded with crystalline sorbitol grains in the form of a powder. Hot air of 70° C. was used to fluidize the powder in a fluid bed. An aqueous solution of sucralose having a sucralose concentration of 10, 20 and 25 g/l was sprayed from above onto the sorbitol powder to make respectively 0.2, 0.4 and 0.5 wt.-% concentration of sucralose in sorbitol.

A first trial was done by spraying water onto the fluid bed of sorbitol in order to achieve a free flowing granulate of sorbitol. About 600 ml was sprayed onto the sorbitol to achieve this (sample 6).

Hereafter the sucralose concentration was calculated in respectively a 0.2, 0.4 and 0.5 wt.-% concentration of sucralose in sorbitol. In Table 1 below, the sucralose content of those three powders are summarized:

TABLE 1

| | | Dry | Dry | Dry | Dry |
|---|---|---|---|---|---|
| | | sucralose + | sorbitol + | sorbitol + | sorbitol + |
| | | 0% | 0.2%/wt | 0.4%/wt | 0.5%/wt |
| Parameter | Unit | sucralose | sucralose | sucralose | sucralose |
| Sucralose | ppm/cp | 0 | 1715 | 3317 | 4200 |
| (dosage) | | | | | |
| SFG | | 6 | 7 | 8, 11 and | 4 12 and |
| sample | | | | 25 | 26 |

In Table 2 below, the sieve analyses are given of the starting product and the respective agglomerates

TABLE 2

| | >53 μm | >100 μm | >150 μm | >250 μm | >400 μm |
|---|---|---|---|---|---|
| Sorbitol as is not | 99.3% | 87.9% | 68.5% | 32.2% | 1.0% |
| aggl. | | | | | |
| Sorbitol | 100% | 96.5% | 88.4% | 61.7% | 13.6% |
| agglomerated on | | | | | |
| a Glatt GPCG5 | | | | | |
| pilot plant as is | | | | | |
| Dry sorbitol + | 100% | 97.0% | 89.0% | 63.5% | 19.6% |
| 0.2%/wt | | | | | |
| sucralose (co- | | | | | |
| agglomerated) | | | | | |
| Dry sorbitol + 0.4%/wt | 100% | 98.3% | 92.4% | 70.3% | 22.6% |
| sucralose | | | | | |
| Dry sorbitol + | 100% | 97.6% | 91.8% | 70.8% | 21.1% |
| 0.5%/wt | | | | | |
| sucralose (co- | | | | | |
| agglomerated) | | | | | |

| | <53 μm | 53-100 μm | 100-150 μm | 150-250 μm | 250-400 μm | >400 μm |
|---|---|---|---|---|---|---|
| Sorbitol as is | 0.7% | 11.4% | 19.4% | 36.4% | 31.2% | 1.0% |
| not aggl. | | | | | | |
| Sorbitol | 0% | 3.5% | 8.0% | 26.4% | 48.1% | 13.6% |
| agglomerated | | | | | | |
| on a Glatt | | | | | | |
| GPCG5 pilot | | | | | | |
| plant as is | | | | | | |
| Dry sorbitol + | 0% | 2.9% | 8.1% | 25.5% | 43.9% | 19.6% |
| 0.2%/wt | | | | | | |
| sucralose (co- | | | | | | |
| agglomerated) | | | | | | |
| Dry sorbitol + | 0% | 1.7% | 5.9% | 22.1% | 47.8% | 22.6% |
| 0.4%/wt | | | | | | |
| sucralose | | | | | | |
| Dry sorbitol + | 0% | 2.4% | 5.8% | 21.1% | 49.6% | 21.1% |
| 0.5%/wt | | | | | | |
| sucralose (co- | | | | | | |
| agglomerated) | | | | | | |

The co-sprayed comparative sample was produced according to a conventional co-spray-drying process.

TABLE 3
Chewing gum, sugar-free formula:

| Ingredients | % | g cp |
|---|---|---|
| Sorbitol powder | 54.0 | 432.0 |
| Sucralose | 0.1 | 0.8 |
| Gum Base | 29.0 | 232.0 |
| Maltitol at 74% ds | 12.0 | 96.0 |
| Mannitol | 2.3 | 18.4 |
| Liquid flavour | 1.4 | 11.2 |
| Dry flavour | 1.0 | 8.0 |
| Soy lecithin | 0.20 | 1.6 |
| Total | 100.0 | 800.0 |

### Procedure Using the Lab Scale Winkworth Z-Blade Mixer:

| Timer up | |
|---|---|
| (min-sec) | Addition steps |
| | 1) turn on the waterbath on 50° C. (have to reach 45-50° C. |
| | in the gum mixer) |
| | 2) microwave the gum base sheets for 3 min (max. 60° C.) |
| | or put the gum base in an oven at 45° C. for min. 15 min |
| 0.00 | 3) add the half of the sorbitol powder + co-agglomerate |
| | sucralose/sorbitol + all of the mannitol powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + Sucralose when added |
| | separately |
| 10.00 | 8) mix until smooth |

TABLE 4
Experimental plan:

| | | product sucralose/sorbitol (g) | | | |
|---|---|---|---|---|---|
| | | | co-agglomerated | | |
| | | | on Glatt GPCG5 | | |
| | | | pilot plant | | |
| | | | Sucralose 0.5% | | |
| | Pure dry | Co-sprayed | (measured later | Sucralose | Final |
| Experiment | Sorbitol (g) | Sucralose 0.417% | 0.420%) | (g) | sucralose % |
| Comp. | 432 | | | 0.8 | 0.1 |
| Sample 1 | | | | | |
| Comp. | 432 | | | 0 | 0 |
| Sample 2 | | | | | |
| Comp. | 241 | 191.8 | | 0 | 0.1 |
| Sample 3 | | | | | |
| Sample 4 | 272.8 | | 160 | 0 | 0.1 |

### Results:

Ranking (7 panelists, 4 samples—Kramer range: 11-24, LSD=9.47)

Sweetness onset time: the rate at which the first signs of sweetness appear The panelists were asked to rank the sample from slower to faster sweetness onset time:

Slower sweetness onset time

| | |
|---|---|
| Comp. | 10 |
| Sample 2 | |
| Comp. | 17 |
| Sample 1 | |
| Comp. | 18 |
| Sample 3 | |
| Sample 4 | 25 |

Faster sweetness onset time

According to the Friedman and the Kramer tests and the LSD value a significant difference was found in sweetness onset time between Comp. Sample 2 chewing gum and Sample 4: Comp. Sample 2 chewing gum had a significantly slower sweetness onset time, indicating that the sweetness appeared later, whereas Sample 4 had a faster sweetness onset time. There was furthermore a tendency that the sample with the sorbitol/sucralose agglomerated combo (Sample 4) allowed for a faster sweetness onset than the spray dried sorbitol/sucralose combo (Comp. Sample 3). This is probably related to the physical distribution of the sucralose in the combo, either as a coating (agglomerated) or as a thoroughly mixed component (spray-dried).

The experiment was carried out taking into account the theoretical sucralose content 0.5% in the Co-agglomerate sample 4 while after dosage the % sucralose was 16% lower than the expected value. Thus, the real sucralose content would have emphasized the phenomenon observed with the Sugar Free Chewing Gum sample 4 containing the Co-agglomerate according to the invention.

1.2 The co-agglomerated samples were produced as Example 1.1.

The Chewing gum sticks were produced according to the following Chewing gum sugar-free recipe:

TABLE 5
Chewing gum sugar-free formula:

| Ingredients | % | g cp |
|---|---|---|
| Sorbitol powder | 54.0 | 432.0 |
| Sucralose | 0.09 | 0.74 |
| Gum Base | 29.0 | 232.0 |
| Maltitol at 74% ds | 12.0 | 96.0 |
| Mannitol | 2.3 | 18.4 |
| Liquid flavour | 1.4 | 11.2 |
| Dry flavour | 1.0 | 8.0 |
| Soy lecithin | 0.20 | 1.6 |
| Total | 100.0 | 800.0 |

### Procedure Using the Lab Scale Winkworth Z-Blade Mixer:

| Timer up | |
|---|---|
| (min-sec) | Addition Steps |
| | 1) turn on the waterbath on 50° C. (have to reach 45-50° C. |
| | in the gum mixer) |
| | 2) microwave the gum base sheets for 3 min (max. 60° C.) |
| | or put the gum base in an oven at 45° C. for min. 15 min |
| 0.00 | 3) add the half of the sorbitol powder + co-agglomerate |
| | sucralose/sorbitol + all of the mannitol powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + Sucralose when added |
| | separately |
| 10.00 | 8) mix until smooth |

TABLE 6
Experimental plan

| | | Sorbitol/sucralose (g) | | | | |
|---|---|---|---|---|---|---|
| | | Co-agglomerated on | | | | |
| | | Glatt GPCG5 | | | | |
| | | pilot plant | | | | |
| | Pure dry | sucralose | sucralose | sucralose | Sucralose | Final |
| Sample | Sorbitol (g) | 0% | 0.1715% | 0.3317% | (g) | sucralose % |
| Comp. 5 | 432 | | | | 0.74 | 0.09 |
| Comp. 6 | | 432 | | | 0.74 | 0.09 |
| 7 | | | 432 | | 0 | 0.09 |
| 8 | | 208 | | 224 | 0 | 0.09 |
| Comp. 9 | | 432 | | | 0 | 0 |

Ranking (8 panelists, 4 samples—Kramer range: 13-27, LSD=10.12)

Sweetness onset time: the rate at which the first signs of sweetness appear. The panelists were asked to rank the sample from slower to faster sweetness onset time:

Slower sweetness onset time

| | |
|---|---|
| Comp. | 11 |
| Sample 9 | |
| Comp. | 15 |
| Sample 6 | |
| Sample 8 | 26 |
| Sample 7 | 28 |

Faster sweetness onset time

According to the Friedman and the Kramer tests and the LSD value a significant difference was found in sweetness onset time. Comp. Samples 6 and 9 had a significantly slower sweetness onset time than Samples 7 and 8.

Sweetness build-up: the rate at which the sweetness is built up to a maximum. The panelists were asked to rank the sample from slower to faster sweetness build-up:

Slower sweetness build-up

| | |
|---|---|
| Comp. | 13 |
| Sample 9 | |
| Comp. | 16 |
| Sample 6 | |
| Sample 7 | 24 |
| Sample 8 | 27 |

Faster sweetness build-up

According to the Friedman and the Kramer tests and the LSD value a significant difference could be found in sweetness build-up between the Comp. Sample 9 chewing gum on the one hand and the samples 7 and 8 on the other hand.

Maximum sweetness level: the intensity of sweetness obtained at the maximum sweetness level (plateau). The panelists were asked to rank the sample from lower (less sweet) to higher (sweeter):

Lower (less sweet)

| | |
|---|---|
| Comp. | 9 |
| Sample 9 | |
| Comp. | 19 |
| Sample 6 | |
| Sample 7 | 24 |
| Sample 8 | 28 |

Higher (sweeter)

According to the Friedman and the Kramer tests and the LSD value a significant difference was found in maximum sweetness level between Comp. Sample 9 chewing gum on the one hand and samples 7 and 8 on the other hand, which were sweeter.

1.3 The co-agglomerated samples were produced as in experiment 1.1

Chewing gum sticks were produced according to the following chewing gum sugar-free recipe.

TABLE 7
Chewing gum sugar-free formula:

| Ingredients | % | G cp |
|---|---|---|
| Sorbitol powder | 54.0 | 432.0 |
| Sucralose | 0.1 | 0.8 |
| Gum Base | 29.0 | 232.0 |
| Maltitol at 74% ds | 12.0 | 96.0 |
| Mannitol | 2.3 | 18.4 |
| Liquid flavour | 1.4 | 11.2 |
| Dry flavour | 1.0 | 8.0 |
| Soy lecithin | 0.20 | 1.6 |
| Total | 100.0 | 800.0 |

### Procedure Using the Lab Scale Winkworth Z-Blade Mixer:

| Timer up | |
|---|---|
| (min-sec) | Addition Steps |
| | 1) turn on the waterbath on 50° C. (have to reach 45-50° C. |
| | in the gum mixer) |
| | 2) microwave the gum base sheets for 3 min (max. 60° C.) |
| | or put the gum base in an oven at 45° C. for min. 15 min |
| 0.00 | 3) add the half of the sorbitol powder + co-agglomerate |
| | sucralose/sorbitol + all of the mannitol powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + Sucralose when added |
| | separately |
| 10.00 | 8) mix until smooth |

TABLE 8
Experimental plan

| | | Sorbitol/sucralose (g) | | | |
|---|---|---|---|---|---|
| | | Co-agglomerated | | | |
| | | on Glatt GPCG5 | | | |
| | | pilot plant | | | Final |
| | Pure dry | sucralose | sucralose | Sucralose | sucralose |
| Sample | Sorbitol (g) | 0.3317% | 0.4200% | (g) | % |
| Comp. 10 | 432.0 | | | 0.8 | 0.1 |
| 11 | 190.8 | 241.2 | | 0 | 0.1 |
| 12 | 241.5 | | 190.5 | 0 | 0.1 |

Ranking (8 panelists, 3 samples—Kramer range: 11-21, LSD=7.84)

Sweetness onset time: the rate at which the first signs of sweetness appears The panelists were asked to rank the samples from slower to faster sweetness onset time:

Slower sweetness onset time

| | |
|---|---|
| Comp. Sample 10 | 10 |
| Sample 12 | 15 |
| Sample 11 | 23 |

Faster sweetness onset time

According to the Friedman and the Kramer tests and the LSD value a significant difference in sweetness onset time was found. The chewing gum with sorbitol+sucralose added separately had a significantly slower sweetness onset than the chewing gum with agglomerated sorbitol/sucralose at 0.3317% sucralose.

The chewing gum with co-agglomerated sorbitol/sucralose with sucralose at 0.3317% sucralose was significantly different from the one with 0.4200% sucralose, which had an intermediate onset between the faster onset sample with sorbitol/sucralose at 0.3317% sucralose and the slower onset sample with sorbitol+sucralose added separately.

Maximum sweetness level: the intensity of sweetness obtained at the maximum sweetness level (plateau)

The panelists were asked to rank the samples from slower (less sweet) to higher (sweeter):

Lower (less sweet)

| | |
|---|---|
| Comp. Sample 10 | 9 |
| Sample 12 | 17 |
| Sample 11 | 22 |

Higher (sweeter)

According to the Friedman and the Kramer tests and the LSD value a significant difference in maximum sweetness could be found. The chewing gum with sorbitol+sucralose added separately had a significantly lower maximum sweetness than both chewing gums with co-agglomerated sorbitol/sucralose according to the invention.

1.4 The top-sprayed samples were produced according to the following protocol: Each run in the fluid bed of the Glatt-pilot (GPCG 5) was loaded with 3000 g of sorbitol powder. Sucralose solution was sprayed onto the powders by top-spray. The incoming air was heated to a temperature between 71 and 73° C. Within 15 minutes 600 ml of sucralose solution was sprayed onto the fluidized powder. During spraying the powder-temperature dropped from 52° C. to 42° C. After spraying of the sucralose solution, the powder was dried for 20 minutes and at the end of the drying cycle, the powder-temperature raised again to a value of 52° C. The water-content of the final dried agglomerates was measured with Karl Fisher and varied between 0.18 and 0.20 wt.-% which is within specification. Three tests were done. First a sorbitol-sucralose agglomerate was made aiming at 0.5 wt.-% sucralose concentration in the agglomerate by spraying with a 25 g/l sucralose-solution. A second test aiming at 0.99 wt.-% sucralose using a 50 g/l sucralose solution and a third test aiming at 1.96 wt.-% with a 200 g/l sucralose solution. A blank-test run by spraying only water onto the sorbitol was also performed.

The bottom-sprayed samples were produced according to the following protocol: In a Procept laboratory fluid bed, operated in the bottom-spray mode, a fluid-bed of sorbitol particles was created by blowing air through a grit. An aqueous sucralose solution was sprayed into the fluid bed from the bottom through a nozzle located in the centre of this grit. The dry sorbitol particles moved in the same direction as the liquid sucralose droplets, forming a coating of sucralose on the surface of the sorbitol grains

The fluid-bed (Procept) was filled with 350 g of sorbitol powder. Hot air of 70° C. was blown in the reactor in such a way a stable fluid-bed was created. Product-temperature was around 65° C.

Above the bottom-screen a tube was put into the fluid-bed and the spraying was done from the bottom. The bottom-screen had also larger openings in the centre and smaller openings at the border. This created an upward flow of powder in the tube, while powders were coming down again outside this tube. This “Wurster” setup was used to result in better coating quality as compared to top-spraying.

In every test, 30 ml of sucralose-solution were sprayed onto the powder at a rate of 2 ml/min. The concentration of the sucralose solution was prepared and changed for every run in such a way that with 30 ml of spraying a final sucralose content of 0.5, 1 and 2 wt.-%, respectively, was achieved.

Powders were made with different ratios sucralose/sorbitol between 0.5 up to 2%. Three top-sprayed sucralose/sorbitol co-agglomerates and four bottom-sprayed co-agglomerates were produced.

1.4.1. The top-sprayed sucralose/sorbitol powders (co-agglomerates) Three top-sprayed co-agglomerated sucralose/sorbitol powders at 3 different sucralose/sorbitol ratios of 0.5, 1.0 and 2.0 wt % were produced as in experiment 1.1 with modifications as shown in Table 9 below. The final sucralose content of those three powders are summarized:

TABLE 9

| | | Sorbitol powder + | | |
|---|---|---|---|---|
| | | 0.5% | 1.0% | 2.0% |
| Parameter | Unit | sucralose | sucralose | sucralose |
| Sucralose | % | 0.42 | 0.85 | 1.75 |
| measured | | | | |

1.4.2. The bottom-sprayed sucralose/sorbitol powders (co-agglomerates)

Four bottom-sprayed co-agglomerated sucralose/sorbitol powders at 4 different sucralose/sorbitol ratios of 0.2, 0.5, 1.0 and 2.0 wt % were produced.

In Table 10 below, the final sucralose content of those four powders are summarized:

TABLE 10

| | | Sorbitol powder + | | |
|---|---|---|---|---|
| Parameter | Unit | 0.5% sucralose | 1.0% sucralose | 2.0% sucralose |
| Sucralose | % | 0.43 | 0.92 | 2.24 |
| measured | | | | |

1.4.3. Effect of the sucralose co-agglomeration and coating on the PSD of the sorbitol powder.

In Table 11 below, the particle size distribution (PSD) of the co-agglomerated sucralose/sorbitol powders is summarized.

TABLE 11

| Co-agglomerate powder | | Particle Size Distribution (%) | | | | | |
|---|---|---|---|---|---|---|---|
| type | % sucralose | >53μ | >150μ | >250μ | >400μ | >600μ | >1000μ |
| Sorbitol powder as is | 0 | 99.9 | 68.5 | 32.2 | 1 | — | — |
| Top-sprayed | 0.42 | 100 | 90.4 | 53.5 | 2.5 | 0.5 | — |
| Sucralose/sorbitol | 0.85 | 100 | 90 | 51.5 | 2 | 0.5 | — |
| | 1.75 | 100 | 82.5 | 43.5 | 2 | 0.7 | — |
| Bottom-sprayed | 0.43 | 100 | 94 | 78.7 | 22 | 4 | — |
| Sucralose/sorbitol | 0.92 | 100 | 90 | 68.7 | 16.7 | 5.3 | — |
| | 2.24 | 100 | 79.3 | 54.7 | 5.3 | 0.6 | — |

It appears that both co-agglomeration technologies induce a shift of the powder characteristics to coarser particles. Such particle size increase is more important when the sucralose is bottom-sprayed on the sorbitol while in Glatt top-spraying technique, 98% of the particles remain below 400μ.

In top-sprayed co-agglomeration the PSD is practically independent from the ratio sucralose/sorbitol.

1.4.4. Chewing gum sticks were produced according to the following chewing gum sugar-free recipe.

TABLE 12
Chewing gum sugar-free formula:

| Ingredients | % | g cp |
|---|---|---|
| Sorbitol powder | 54.0 | 432.0 |
| Sucralose | 0.1 | 0.8 |
| Gum Base | 29.0 | 232.0 |
| Maltitol at 74% ds | 12.0 | 96.0 |
| Mannitol | 2.3 | 18.4 |
| Liquid flavour | 1.4 | 11.2 |
| Dry flavour | 1.0 | 8.0 |
| Soy lecithin | 0.20 | 1.6 |
| Total | 100.0 | 800.0 |

### Procedure Using the Lab Scale Winkworth Z-Blade Mixer:

| Timer up | |
|---|---|
| (min-sec) | Addition Steps |
| | 1) turn on the waterbath on 50° C. (have to reach 45-50 20 C. |
| | in the gum mixer) |
| | 2) microwave the pallets gum base sheets for 3 min (max. |
| | 60° C.) or put the pullets gum base in an oven at 45° C. |
| | for min. 15 min |
| 0.00 | 3) add the half of the sorbitol powder + top- or bottom- |
| | sprayed agglomerate sucralose/sorbitol + all of the mannitol |
| | powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + Sucralose when added |
| | separately |
| 10.00 | 8) mix until smooth |

SFG with bottom-sprayed sucralose/sorbitol co-agglomerate and the reference sucralose and sorbitol added separately

TABLE 13

| | | Coated or Bottom- | | | | |
|---|---|---|---|---|---|---|
| Sugar | Pure | sprayed sucralose/sorbitol | | | | Final |
| free | dry | agglomerate (g) | | | | sucralose |
| gum | Sorbitol | sucralose/sorbitol ratio | | | Sucralose | % in the |
| sample | (g) | 0.43% | 0.92% | 2.24% | (g) | SFG |
| 13 | 540 | | | | 1.0 | 0.1 |
| 14 | 309 | 232 | | | — | 0.1 |
| 15 | 433 | | 108 | | — | 0.1 |
| 16 | 494 | | | 47 | — | 0.1 |

TABLE 14
SFG with top-sprayed sucralose/sorbitol co-agglomerate

| Sugar | Pure | top-sprayed sucralose/ | | | | Final |
|---|---|---|---|---|---|---|
| free | dry | sorbitol co-agglomerate (g) | | | | sucralose |
| gum | sorbitol | Sucralose/sorbitol ratio | | | Sucralose | % |
| sample | (g) | 0.42% | 0.85% | 1.75% | (g) | in the SFG |
| 17 | 305 | 236 | | | — | 0.1 |
| 18 | 423 | | 118 | | — | 0.1 |
| 19 | 484 | | | 57 | — | 0.1 |

For all three ratios, both the top-sprayed co-agglomerated and the bottom-sprayed co-agglomerated sucralose/sorbitol chewing gums showed a significant or near significantly more immediate sweetness onset than the reference chewing gum with sucralose and sorbitol added separately (comp. sample 13).

Additionally, at least 2 or all 3 ratios of the bottom-sprayed co-agglomerated sucralose/sorbitol chewing gum had a significantly or near significantly faster sweetness build-up and higher maximum sweetness level than the reference chewing gum with sucralose and sorbitol added separately (comp. sample 13).

### Example 2

### High Sucralose Concentration Sorbitol Co-Agglomerate Vs. Sucralose, Sorbitol Added Separately

Co-agglomerates were produced as described in experiment 1.1, but with higher sucralose/sorbitol ratio at 2%, 5% and 10% to check the technical feasibility of the production of high ratio sucralose/sorbitol powders and simplify for the chewing gum producer the handling of co-agglomerated sucralose/sorbitol. Indeed an increase of the sucralose/sorbitol ratio from 2% to 10% allows to SFG producer to reduce the quantity of such new ingredient from 5% to 1% in the final SFG recipe. The results obtained with SFG comprising such high ratio co-agglomerated sucralose/sorbitol combos clearly demonstrate that the onset sweetness perceived with co-agglomerated sucralose/sorbitol according to the invention at high sucralose/sorbitol ratio is faster than in comparative chewing gums produced by separately adding the same amount of sucralose and sorbitol.

TABLE 15

| Sucralose source | Co-agglomerate sucralose/sorbitol | | |
|---|---|---|---|
| Co-agglomerated | 2% | 5% | 10% |
| sucralose/sorbitol | | | |
| ratio | | | |
| Quantity of co- | 5% | 2% | 1% |
| agglomerate in | | | |
| the SFG recipe | | | |
| Final sucralose % | 0.1% | | |
| in the SFG | | | |
| Sample | 20 | 21 | 22 |

### Example 3

### Maltitol as the Polyol

Further experiments have been carried out producing chewing gum sticks containing sucralose combined with sorbitol and/or maltitol, all added separately or sucralose added as co-agglomerated on maltitol powder.

The co-agglomerated samples were produced according to the protocol explained in the Experiment 1.1 for the co-agglomerate of sucralose on sorbitol. The sucralose content on the sorbitol powder was measured at 0.33%.

With regard to the co-agglomerate of sucralose on maltitol the protocol is similar. The co-agglomerated sample 24 was produced by top-spraying an aqueous solution of sucralose onto a fluid-bed of maltitol powder in a Glatt GPCG5 pilot plant batch reactor. The reactor was loaded with 3 kg maltitol powder. Top-spraying is a widely used technology for making (partial) coatings. According to the following protocol the reactor was loaded with crystalline maltitol grains in the form of a powder. Hot air was used to fluidize the powder in a fluid bed. An aqueous solution of sucralose was sprayed from above onto the maltitol powder. The final sucralose content on the maltitol powder was measured at 2.86%.

A sensory test was set up to check possible differences in sweetness onset and maximum sweetness level.

That experimental plan allowed the applicants to determine if co-agglomerated sucralose-maltitol changes the profile of the chewing gum versus sucralose, maltitol and sorbitol added separately.

The conclusions of the sensory panel analysis carried out show that the sweetness profile of the chewing gum with agglomerated sucralose-maltitol differs from the profile of the chewing gum with sucralose+maltitol+sorbitol added separately. The stick containing co-agglomerated sucralose-maltitol has a significant faster sweetness onset, a significant higher maximum sweetness level and a tendency towards a longer sweetness lingering.

It can therefore be extrapolated that whatever the polyol powder used in the co-agglomeration of sucralose on the dry polyol grain surface, it influences the gum sweetness profile changing to faster sweetness onset and a higher maximum sweetness.

The Chewing gum sticks were produced according to the following Chewing gum sugar-free recipe:

TABLE 16
Chewing gum sugar-free formula:

| | Sugar free gum sample | | | | | |
|---|---|---|---|---|---|---|
| | Sample with | | | | | |
| | all ingredients | | Sample with sucralose | | | |
| | added | | co-agglomerated with | | | |
| | Separately | | Maltitol | | Sorbitol | |
| | sample n^{o} | | | | | |
| | 23 | | 24 | | 25 | |
| Ingredients | % | g cp | % | g cp | % | g cp |
| Sorbitol powder | 50.6 | 506 | 50.6 | 506 | 20.4 | 204 |
| Maltitol powder | 3.4 | 34 | — | — | 3.4 | 34 |
| Co-aggl. sucralose/ | — | — | 3.5 | 35 | — | — |
| maltitol (ratio | | | | | | |
| sucralose/maltitol | | | | | | |
| 2.86%) | | | | | | |
| Co-aggl. sucralose/ | — | — | — | — | 30.3 | 303 |
| sorbitol (ratio | | | | | | |
| sucralose/sorbitol | | | | | | |
| 0.33%) | | | | | | |
| Sucralose added | 0.1 | 1 | — | — | — | — |
| separately | | | | | | |
| Gum Base | 29.0 | 290 | 29.0 | 290 | 29.0 | 290 |
| Maltitol at 74% ds | 12.0 | 120 | 12.0 | 120 | 12.0 | 120 |
| Mannitol | 2.3 | 23 | 2.3 | 23 | 2.3 | 23 |
| Liquid flavour | 1.4 | 14 | 1.4 | 14 | 1.4 | 14 |
| Dry flavour | 1.0 | 10 | 1.0 | 10 | 1.0 | 10 |
| Soy lecithin | 0.2 | 2 | 0.2 | 2 | 0.2 | 2 |
| Total | 100.0 | 1000 | 100.0 | 1000 | 100.0 | 1000 |
| Total sucralose | 10.1 | | 0.1 | | 0.1 | |

Procedure using the lab scale Winkworth Z-blade mixer:

| Timer up | |
|---|---|
| (min-sec) | Addition Steps |
| | 1) turn on the waterbath on 50° C. (have to reach 45-50° C. in |
| | the gum mixer) |
| | 2) microwave the gum base sheets for 3 min (max. 60° C.) |
| | or put the gum base in an oven at 45° C. for min. 15 min |
| 0.00 | 3) add the half of the sorbitol powder + co-agglomerate |
| | sucralose/polyol or maltitol powder + all of the mannitol |
| | powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + sucralose when added |
| | separately |
| 10.00 | 8) mix until smooth |

Ranking (8 Panelists, 3 samples, Kramer range 11-21, LSD=7.84)

Sweetness onset: The speed at which sweetness (of any type) is first sensed. The panelists were asked to rank the samples from “immediate” to “delayed” sweetness onset:

Immediate sweetness onset

| | |
|---|---|
| Sample 24 | 10 |
| Sample 25 | 17 |
| Comp. | 21 |
| Sample 23 | |

Delayed sweetness onset

According to the Friedman and Kramer tests and the LSD value a significant difference in sweetness onset was found. The chewing gum with agglomerated maltitol/sucralose had a significantly faster sweetness onset than the chewing gum with sucralose, maltitol, sorbitol added separately.

Maximum sweetness level: The panelists were asked to rank the samples from lower to higher maximum sweetness intensity:

Lower (less sweet)

| | |
|---|---|
| Comp. | 10 |
| Sample 23 | |
| Sample 25 | 15 |
| Sample 24 | 23 |

Higher (sweeter)

According to the Friedman and the Kramer tests and the LSD value a significant difference in maximum sweetness was found. The chewing gum with agglomerated maltitol/sucralose had a significantly higher maximum sweetness level than the chewing gum with sucralose, maltitol, sorbitol added separately. According to the LSD (7.84), the difference between the chewing gum with agglomerated sorbitol/sucralose and with agglomerated maltitol/sucralose was also significant, the latter showing higher maximum sweetness.

### Example 4

### Perception of Mintiness Intensity/Comparison Between Sucralose/Sorbitol Conventional Combo, Sucralose/Sorbitol Co-Agglomerate and Aspartame/Acesulfame-K Combo

The aim of this experiment was to check whether the sweetness and mint flavour profiles are different for Sugar Free Chewing Gum with aspartame/acesulfame-K (Asm/AcK) from chewing gum with separately added sucralose or co-agglomerated sucralose/sorbitol.

In a first session, among Sugar Free Chewing Gum with separately added sucralose ranging from 0.1% to 0.2% a chewing gum was selected lying closest in maximal sweetness level to the Sugar Free Chewing Gum with Asm/AcK (0.18%/0.12%), which has been chosen as representative of conventional commercial products. The sucralose content of the selected Sugar Free Chewing Gum (SFG) was 0.18%.

In a second test, the selected chewing gum with 0.18% separately added sucralose was compared to a Sugar Free Chewing Gum with Asm/AcK (0.18%/0.12%) and a Sugar Free Chewing Gum with co-agglomerated sucralose/sorbitol containing 0.18% sucralose in the final chewing gum.

The data reported Time Intensity sensory analysis of chewing gum showed that the gum containing co-agglomerated sucralose-sorbitol had a faster onset on sweetness perception and mintiness perception. Moreover, the co-agglomerated sucralose-sorbitol gum reached a parallel higher sweetness perception and mint intensity perception during chewing.

The Chewing gum sticks were produced according to the following Chewing gum sugar-free recipe.

TABLE 17
Chewing gum sugar-free formula:

| | Sugar free gum sample | | | | | |
|---|---|---|---|---|---|---|
| | | | | | Sample with | |
| | | | | | top-sprayed | |
| | | | | | sucralose/ | |
| | | | | | sorbitol co- | |
| | | | | | agglomerate | |
| | | | Sample with | | (g) | |
| | Sample with | | sucralose + | | sucralose/ | |
| | Asm/Ace-K | | sorbitol added | | sorbitol | |
| | (0.18%/0.12%) | | separately | | ratio 0.42% | |
| | Sample n^{o} | | | | | |
| | 26 | | 27 | | 28 | |
| Ingredients | % | g cp | % | g cp | % | g cp |
| Sorbitol powder | 53.8 | 538 | 53.92 | 539.2 | 11.2 | 112 |
| Co-aggl. | — | — | — | — | 42.9 | 429 |
| sucralose/ | | | | | | |
| sorbitol (ratio | | | | | | |
| sucralose/sorbitol | | | | | | |
| 0.42%) | | | | | | |
| Sucralose added | — | — | 0.18 | 1.8 | — | — |
| separately | | | | | | |
| Aspartame | 0.18 | 1.8 | — | — | — | — |
| Acesulfame-K | 0.12 | 1.2 | — | — | — | — |
| Gum Base | 29.0 | 290 | 29.0 | 290 | 29.0 | 290 |
| Maltitol at | 12.0 | 120 | 12.0 | 120 | 12.0 | 120 |
| 74% ds | | | | | | |
| Mannitol | 2.3 | 23 | 2.3 | 23 | 2.3 | 23 |
| Liquid flavour | 1.4 | 14 | 1.4 | 14 | 1.4 | 14 |
| Dry flavour | 1.0 | 10 | 1.0 | 10 | 1.0 | 10 |
| Soy lecithin | 0.2 | 2 | 0.2 | 2 | 0.2 | 2 |
| Total | 100.0 | 1000 | 100.0 | 1000 | 100.0 | 1000 |
| Total sucralose | — | — | 0.18 | | 0.18 | |

Procedure using the lab scale Winkworth Z-blade mixer:

| Timer up | |
|---|---|
| (min-sec) | Addition Steps |
| | 1) turn on the waterbath on 50° C. (have to reach 45-50° C. |
| | in the gum mixer) |
| | 2) microwave the gum base sheets for 3 min (max. 60° C.) |
| | or put the gum base in an oven at 45° C. for min. 15 min |
| 0.00 | 3) add the half of the sorbitol powder + co-agglomerate |
| | sucralose/sorbitol + all of the mannitol powder |
| 0.00 | 4) start mixing on full power mode |
| 0.30 | 5) add the gum base + soy lecithin |
| 2.30 | 6) add the remaining sorbitol powder + maltitol syrup |
| 5.30 | 7) add flavours dry & liquid + Sucralose when added |
| | separately or Asm + Ack |
| 10.00 | 8) mix until smooth |

The following three chewing gum samples were produced and used for the sensory analysis of this example:

Comparative sample 26 comprising Asm/Ace-K (0.18%/0.12% in the final product)
Comparative sample 27 comprising isolated sucralose (0.18% in the final product)
Sample 28 comprising a co-agglomerate at 0.18% sucralose in the final product

Comparative samples 26 and 27 were chosen to have the same maximal sweetness perception. All samples were cut in half before assessment.

Time-intensity analysis: Using ten trained panelists the perceived sweet and mint intensity were measured from chewing gum sticks made with sucralose+sorbitol added separately or co-agglomerated. The panelists underwent a period of training before the proper assessment of the samples. The training involved getting accustomed to a time-intensity line scale (0-100) on a computer screen with the value 100 being set at the sweetness and mintiness perception of a 0.2% sucralose sample. For the main test, the panelists were asked to rate the perceived mintiness and sweetness (not at the same time) of the gum samples over 10 minutes. Each panelist received eight replicates of each sample (four for assessing mintiness and four for assessing sweetness). At the end of the testing a total of 240 time-intensity curves were generated from the ten panelists, and the data was analyzed using FIZZ sensory software.

The individual time-intensity curves revealed that all panelists rated consistently. Using the FIZZ software the curves were first smoothed to remove the peaks generated from the panelists moving the mouse over the ten minutes. Once smoothed, all the curves from each panelist were combined and statistical analysis provided information such as Imax (maximum intensity observed), Tmax (the time to maximum intensity) and SimInc (the maximum slope measured in the increasing phase of the curve).

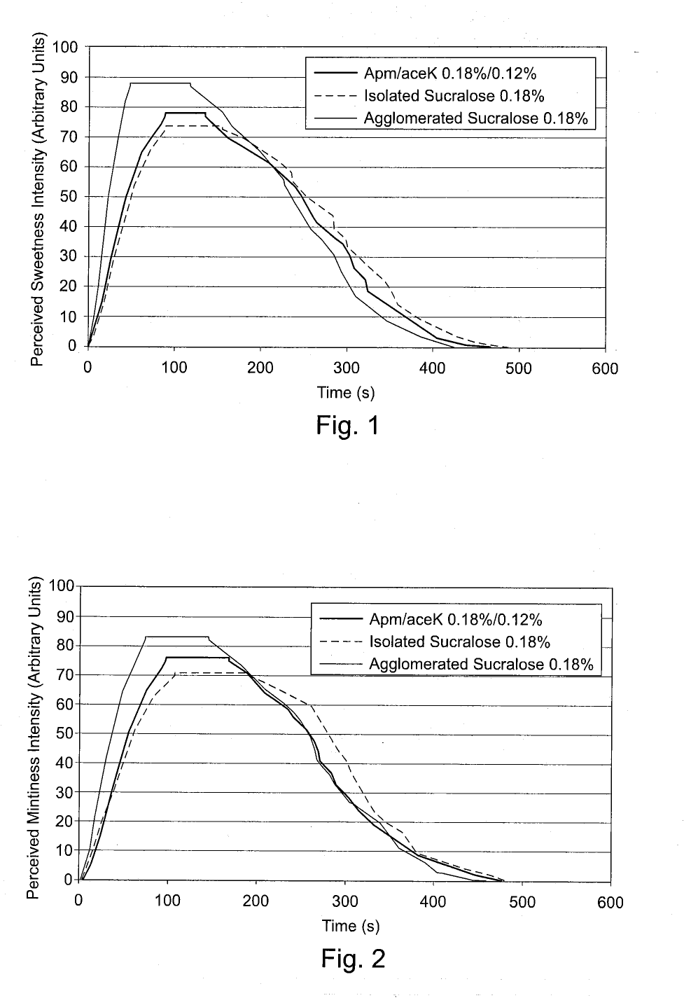

The average curves for each of the three samples are shown in FIGS. 1 and 2. The results are shown in Table 18 below:

TABLE 18

| | Sweetness perception | | | Mintiness perception | | |
|---|---|---|---|---|---|---|
| | | | SimInc | | | SimInc |
| | Imax | | [intensity | Imax | | [intensity |
| | [arbitrary | Tmax | units per | [arbitrary | Tmax | units per |
| Sample | units] | [sec] | second] | units] | [sec] | second] |
| Comp. 26 | 78.7 | 85.7 | 2.6 | 76.0 | 96.6 | 2.2 |
| Comp. 27 | 72.9 | 89.9 | 2.1 | 71.1 | 109.4 | 1.8 |
| 28 | 88.0 | 48.1 | 3.5 | 83.0 | 73.0 | 3.0 |

The results show that the gum containing the co-agglomerate according to the invention is statistically (using a LSD and Tukey test at the 95% confidence level) different from the isolated sucralose and aspartame/acesulfame K gum sample. The differences were found in both the perceived sweetness and mintiness. The co-agglomerate sucralose gum reached a higher sweet and mint intensity (Imax) during chewing. It had also a significantly faster onset of sweetness and mintiness and reached a maximum intensity significantly faster (as shown by the short Tmax and high SimInc values). These differences can also be clearly seen in FIGS. 1 and 2.

The similar sweetness and mintiness profiles furthermore support the surprising finding by the inventors that the co-agglomeration of sucralose on sorbitol induces changes in the sweetness perception profiles. Moreover, the invention demonstrates clearly that the aroma perception is related to the sweetness perception profile.

### Example 5

### Sweetness Perception Vs. Sucralose Concentration in Saliva; Aroma, Taste and Sensory Analysis

A liquid mass spectrometry method was developed to measure sucralose, sorbitol and maltitol in saliva of panelists. Comparative samples 26, 27 and sample 28 were tested on six trained panelists. It was found that the sweetener's release profiles did not differ significantly, irrespective from the samples and its particular sweetener combo. From this follows that the surprising acceleration of sweetness and mintiness perception in the onset phase experienced with the chewing gums of the invention, as well as the maximum intensity of sweetness and mintiness perception are no simple function of the sweetener release and the sweetener's concentration in the saliva.

This set of experiments has shown that there is a definite effect of sucralose sweetness on the perceived mintiness of chewing gum. Just a small amount in the gum can dramatically increase the duration of the mint intensity that the consumer perceives, and it also has the advantage that it has no calorific value.

### Example 6

### Extended Sweetness Release from Bentonite

Chewing gum, a semi-crystalline, viscoelastic compound, is an emulsion comprised of a gum base, sweeteners, fillers, softeners, and flavors. The gum base and some of the flavorings are insoluble in water; the other compounds, including the sweeteners are soluble in water and are extracted from the gum during chewing. As a result, the sweetness disappears quickly while the flavor may linger longer, giving an unbalanced flavor profile. Encapsulating sucralose with silica/clay carrier agents can change the colloidal properties of the sucralose and extend the sweetness profile compared to non-encapsulated sucralose. The goal of the following experiments was to examine several novel sucralose delivery systems for extending sweetness in chewing gum.

### 6.1 Chewing Gum Preparation:

Sucralose (micronized) was previously processed using different agglomeration technologies and different silica substrates. Initial screening work indicated the potential for extended sweetness release when sucralose is processed with bentonite and other substrates. To further screen these samples, they were tested in a chewing gum application. Six batches of chewing gum were prepared, each containing 1000 ppm sucralose based on the theoretical sucralose concentration for the sucralose ingredients described in Table 19 below. A seventh batch, which did not contain any sucralose, and served as the “blank,” was also prepared. Chewing gum was prepared in 1500 gram batch sizes using a Littleford twin sigma blade gum mixer at the University of Wisconsin-Madison. Formulas are given in Table 20.

TABLE 19
Sucralose Samples Evaluated for Extended Sweetness

| Sucralose Sample | Description | % Sucralose |
|---|---|---|
| Micronized | Micronized sucralose | 100.00 |
| Sample G | Sucralose absorbed into bentonite, | 9.90 |
| | co-agglomerated with sorbitol | |
| Sample I | Sucralose absorbed into bentonite, | 25.30 |
| | granulated on top of a bentonite seed | |
| Sample J | Sample I sprayed with a thin guar | 25.30 |
| | gum coating | |
| Sample O | Spray-dried sucralose absorbed into | 71.40 |
| | bentonite | |
| Co-agglomerated | Co-agglomerated sorbitol, sucralose | 11.00 |
| sucralose/sorbitol | | |
| (EU) | | |

TABLE 20
Chewing Gum Formulas

| | Percent | | | | | | |
|---|---|---|---|---|---|---|---|
| | | | | | | | Sample |
| Ingredient | Blank | Micronized | Sample G | Sample I | Sample J | Sample O | EU |
| Crystalline sorbitol, Sorbogem | 55.38 | 55.28 | 54.35 | 54.98 | 54.98 | 55.24 | 54.45 |
| 712¹ | | | | | | | |
| Chewing gum base, Eurodent T- | 29.74 | 29.74 | 29.74 | 29.74 | 29.74 | 29.74 | 29.74 |
| RO² | | | | | | | |
| Maltitol syrup, MALTISWEET | 12.31 | 12.31 | 12.31 | 12.31 | 12.31 | 12.31 | 12.31 |
| 3145¹ | | | | | | | |
| Mannitol powder¹ | 2.36 | 2.36 | 2.36 | 2.36 | 2.36 | 2.36 | 2.36 |
| Soy lecithin, Yellkin TS³ | 0.21 | 0.21 | 0.21 | 0.21 | 0.21 | 0.21 | 0.21 |
| Sucralose, micronized | 0.00 | 0.10 | 0.00 | 0.00 | 0.00 | 0.00 | 0.00 |
| Sucralose, sample G | 0.00 | 0.00 | 1.04 | 0.00 | 0.00 | 0.00 | 0.00 |
| Sucralose, sample I | 0.00 | 0.00 | 0.00 | 0.41 | 0.00 | 0.00 | 0.00 |
| Sucralose, sample J | 0.00 | 0.00 | 0.00 | 0.00 | 0.41 | 0.00 | 0.00 |
| Sucralose, sample O | 0.00 | 0.00 | 0.00 | 0.00 | 0.00 | 0.14 | 0.00 |
| Sucralose, co-agglomerated with | 0.00 | 0.00 | 0.00 | 0.00 | 0.00 | 0.00 | 0.93 |
| sorbitol (EU) | | | | | | | |
| TOTAL | 100.00 | 100.00 | 100.00 | 100.00 | 100.00 | 100.00 | 100.00 |

PROCEDURE:
1. Place pre-weighed gum base in a 45-50° C. (110-120° F.) for at least 30 minutes prior to use to soften the gum base.
2. To the pre-heated gum mixer (45-50° C.), add about half of the sorbitol powder and all of the mannitol power.
3. Start mixing.
4. Add the gum base and soy lecithin to the mixer.
5. Mix for about 1-2 minutes. Stop the mixer and make sure all of the gum base and other ingredients are incorporated.
6. Add the remaining sorbitol powder and maltitol syrup and start the mixer. Mix for 1-2 minutes and check again that all the ingredients are well incorporated.
7. Add the sucralose and any flavors to the mixer. Mix for 1-2 minutes or until smooth and well-incorporated.
8. Laminate the gum to 3 mm thickness (mannitol may be spread on the surface of the gum to help with stickiness, if needed).
9. Cut the gum into 3.0 ± 0.05 gram pieces. Wrap in wax paper or foil (be sure that the gum has cooled to room temperature before wrapping). Place wrapped pieces in foil bag and seal.
10. Allow the gum to rest for at least 24 hours before evaluating.
¹Corn Products, Specialty Ingredients division
²Eurobase
³ADM

### 6.2 Sensory Evaluation:

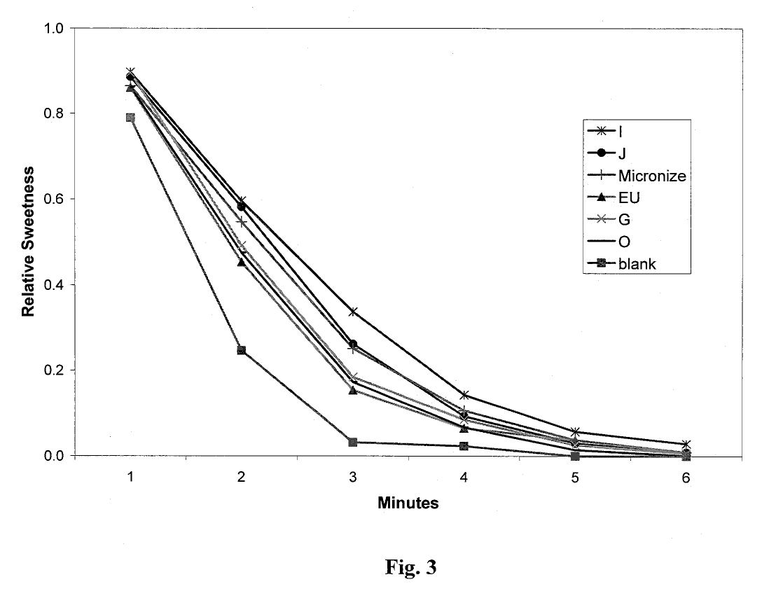

Ten panelists were instructed in magnitude estimation with sucrose solutions, in which panelists estimated the magnitude of increase or decrease in a sensory attribute relative to a reference. In separate tests, panelists were instructed to note the maximum sweetness of chewing gum samples, usually between 20 and 30 seconds of chewing, and to estimate the amount of sweetness remaining at 1 through 6 minutes, relative to the maximum sweetness. Chewing rate was controlled with a metronome at 72 beats per minute. The panelists were served a 3.0±0.05 gram piece of gum wrapped in waxed paper in a soufflé cup labeled with a random three-digit code. The products were evaluated in a randomized order in triplicate over 4 days. Each panelist had their own presentation order. Each panelist evaluated 5 samples per day except for the 4^{th} day when they evaluated 6 samples. Evaluation sessions were one hour per day. Panelists estimated the time to maximum sweetness for each sample in duplicate on the 5^{th} day. Bottled water and unsalted crackers were available before the testing and between samples for the panelists to clear their palates. The sweetness profiles obtained by these methods are shown in Tables 21-22 and FIG. 3.

TABLE 21
Sweetness Relative To Maximum Sweetness

| Sucralose | 1 | 2 | 3 | 4 | 5 | 6 |
|---|---|---|---|---|---|---|
| Sample | minute | minutes | minutes | minutes | minutes | minutes |
| I | 0.90^{a} | 0.60^{a} | 0.34^{a} | 0.14^{a} | 0.06^{a} | 0.03^{a} |
| J | 0.89^{a} | 0.58^{a} | 0.26^{b} | 0.09^{ab} | 0.03^{abc} | 0.01^{ab} |
| Micronized | 0.86^{a} | 0.55^{ab} | 0.25^{b} | 0.11^{ab} | 0.04^{ab} | 0.01^{ab} |
| G | 0.90^{a} | 0.49^{bc} | 0.18^{c} | 0.08^{b} | 0.03^{abc} | 0.01^{b} |
| O | 0.86^{a} | 0.48^{c} | 0.17^{c} | 0.07^{bc} | 0.01^{bc} | 0.00^{b} |
| EU | 0.86^{a} | 0.45^{c} | 0.15^{c} | 0.07^{bc} | 0.04^{ab} | 0.01^{ab} |
| blank | 0.79^{b} | 0.25^{d} | 0.03^{d} | 0.02^{c} | 0.00^{c} | 0.00^{b} |

^{ab}means in a column followed by the same letter are not significantly different α = 0.05

TABLE 22
Time to Maximum Sweetness

| Sucralose | |
|---|---|
| Sample | Time (s) |
| EU | 42 |
| Micronized | 39 |
| J | 39 |
| O | 38 |
| I | 37 |
| G | 36 |
| blank | 35 |

Sample I, made with sucralose absorbed into bentonite and granulated on top of a bentonite seed, maintained a higher sweetness level than the sample made with micronized sucralose for up to 4 minutes. Sample J, which was Sample I coated with a thin layer of guar gum maintained the same sweetness level as the sample made with micronized sucralose. All of the other samples maintained sweetness less well than the sample made with micronized sucralose. Chewing gum with sucralose co-agglomerated with sorbitol had faster sweetness release than the control gum, which may be beneficial for chewing gums with quick flavor onset such as “flavor burst” gums.

These samples were initially hard and took over half a minute to soften. This is reflected in the time it took to reach maximum sweetness. There were no significant differences in the time to maximum sweetness.

### Example 7

### Effects of Hydrocolloid Coatings on Sweetness

These experiments are an extension of Example 6, where it was shown that a bentonite carrier could be used to extend the release of sucralose in chewing gum. The experiments narrow the list of sustained release candidates to a granulated product with a hydrocolloid coating. The effect of particle size/density on the sweetness profile of an agglomerated sucralose/sorbitol product was also explored.

### 7.1 Materials

| | |
|---|---|
| Bentonite | BPM Minerals; National Premiom WT 325 |
| Sucralose | Tate & Lyle; DFF-1 |
| Sorbitol | Corn Products; Sorbogem 712 |

### 7.2 Sustained Release Sample Preparation.

Granulation was carried out on the Glatt ProCell 5 with the bottom-spray configuration on the AGT insert. An aqueous mixture of 10% bentonite and 22.5% sucralose was hydrated in a sonic bath for 1 hour and used as the spray. The bed consisted of 18 micron bentonite particles.

Coating was carried out on the Glatt ProCell 5 with the bottom-spray configuration on the AGT insert. The bed consisted of material from the granulation process, with a spray of 0.5% hydrocolloid solution.

### 7.3 Agglomerated Sorbitol Preparation.

Agglomerated sucralose/sorbitol products were produced on the Glatt ProCell 5 in the top-spray configuration on the GF insert. The bed consisted of Corn Products Sorbogem 712, and the spray was a 35% sucralose solution

Product compositions are summarized in Table 24.

TABLE 24
Experimental Sucralose Samples Evaluated for Extended Sweetness

| | | Spray | | | Final |
|---|---|---|---|---|---|
| | | Fraction | | (g) | Fraction |
| R | Granulated Run 1 Aug. 25, 2008 | | Spray | 700 | |
| 7145-112 | GF Insert, bottom spray | | Bed | 500 | 0.69 |
| | Bentonite Bed | 0.1 | Bentonite | 70 | 0.10 |
| | | 0.225 | Sucralose | 157.5 | 0.22 |
| | | | Total | 727.5 | 1 |
| S | Granulated Run 2 Aug. 26, 2008 | | Spray | 621 | |
| 7145-113 | GF Insert, bottom spray | | Bed | 500 | 0.71 |
| | Bentonite Bed | 0.1 | Bentonite | 62.1 | 0.09 |
| | | 0.225 | Sucralose | 139.725 | 0.20 |
| | | | Total | 701.825 | 1 |
| T | S + Guar Aug. 26, 2008 | | Spray | 594 | |
| 7145-113 | GF Insert, bottom spray | | Bed | 150 | |
| | S Product Bed | | Bentonite | 120.1365 | 0.79 |
| | | | Sucralose | 29.8635 | 0.20 |
| | | 0.005 | Guar | 2.97 | 0.02 |
| | | | Total | 152.97 | 1 |
| U | S + I-Carrageenan Aug. 27, 2008 | | Spray | 600 | |
| 7145-114 | GF Insert, bottom spray | | Bed | 150 | |
| | S Product Bed | | Bentonite | 120.1365 | 0.79 |
| | | | Sucralose | 29.8635 | 0.20 |
| | | 0.005 | I-Carrageenan | 3 | 0.02 |
| | | | Total | 153 | 1 |
| V | R + Gum Arabic Aug. 27, 2008 | | Spray | 600 | |
| 7145-114 | GF Insert, bottom spray | | Bed | 150 | |
| | R Product Bed | | Bentonite | 120.1365 | 0.79 |
| | | | Sucralose | 29.8635 | 0.20 |
| | | 0.005 | Gum Arabic | 3 | 0.02 |
| | | | Total | 153 | 1 |
| W | Low Density Sorbitol Blend | | Sucralose | | 0.11 |
| 7145-115 | AGT Insert, top spray | | Sorbitol | | 0.89 |
| | | | | | 1 |
| X | High Density Sorbitol Blend | | Sucralose | | 0.11 |
| 7145-115 | AGT Insert, top spray | | Sorbitol | | 0.89 |
| | | | | | 1 |

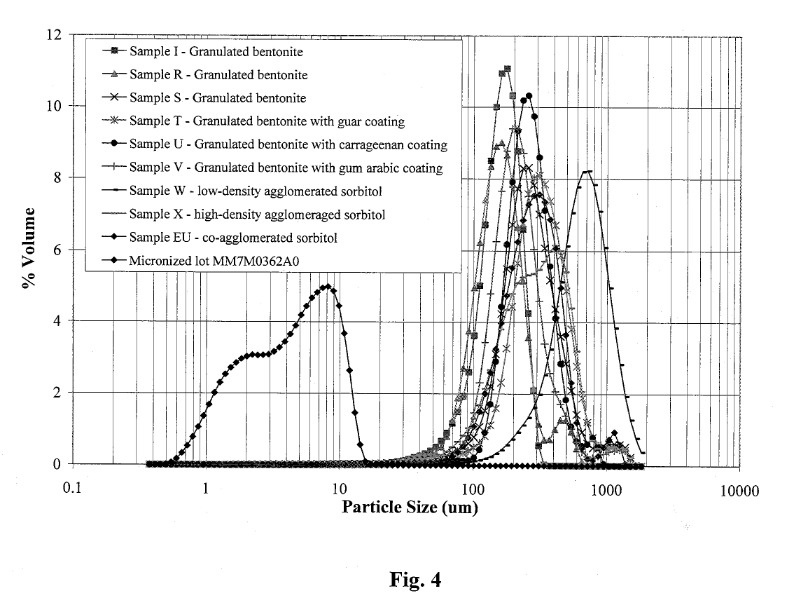

Particle size distribution of each experimental sucralose sample listed in Table 24 was analyzed on the Beckman LS 13 320 Laser Diffraction Particle Size Analyzer using the tornado powder system with the Standard Operating Procedure (SOP) for “dry powder” (Table 25; FIG. 4).

TABLE 25
Mean Particle Size of the Experimental Sucralose Samples

| | | | Mean |
|---|---|---|---|
| Sucralose | | % | Particle |
| Sample | Description | Sucralose | Size (μm) |
| Micronized | Micronized sucralose | 100.00 | 5.31 |
| Sample EU | Sucralose absorbed into bentonite, | 9.90 | 323.98 |
| | co-agglomerated with sorbitol | | |
| Sample I | Sucralose absorbed into bentonite, | 25.30 | 169.25 |
| | granulated on top of a bentonite | | |
| | seed | | |
| Sample R | Sucralose absorbed into bentonite, | 21.65 | 183.62 |
| | granulated on top of a bentonite | | |
| | seed | | |
| Sample S | Sucralose absorbed into bentonite, | 19.91 | 323.56 |
| | granulated on top of a bentonite | | |
| | seed | | |
| Sample T | Sample S coated with a thin layer | 19.52 | 370.18 |
| | of guar gum¹ | | |
| Sample U | Sample S coated with a thin layer | 19.52 | 293.16 |
| | of iota carrageenan² | | |
| Sample V | Sample R coated with a thin layer | 19.52 | 265.39 |
| | of gum arabic³ | | |
| Sample W | Micronized sucralose co- | 11.00 | 722.39 |
| | agglomerated with sorbitol, low | | |
| | density agglomerate | | |
| Sample X | Micronized sucralose co- | 11.00 | 353.93 |
| | agglomerated with sorbitol, high | | |
| | density agglomerate | | |

¹Received from TLCI
²VISCARIN SD 389 lot no. 40814050 from FMC Biopolymers
³FIBERGUM from Colloides Naturels International

### 7.4 Chewing Gum Preparation.

Ten batches of chewing gum were prepared, each containing 1000 ppm sucralose based on the theoretical sucralose concentration for the sucralose ingredients described in Table 24. Chewing gum was prepared in 1500 gram batch sizes using a Littleford twin sigma blade gum mixer at the University of Wisconsin-Madison. Formulas are given in Table 26.

TABLE 26
Chewing Gum Formulas

| | Percent | | | | |
|---|---|---|---|---|---|
| Ingredient | Micronized | Sample EU | Sample I | Sample R | Sample S |
| Crystalline | 55.28 | 54.45 | 54.98 | 54.91 | 54.87 |
| sorbitol, | | | | | |
| Sorbogem 712¹ | | | | | |
| Chewing gum | 29.74 | 29.74 | 29.74 | 29.74 | 29.74 |
| base, Eurodent | | | | | |
| T-RO² | | | | | |
| Maltitol syrup, | 12.31 | 12.31 | 12.31 | 12.31 | 12.31 |
| MALTISWEET 3145¹ | | | | | |
| Mannitol | 2.36 | 2.36 | 2.36 | 2.36 | 2.36 |
| powder¹ | | | | | |
| Soy lecithin, | 0.21 | 0.21 | 0.21 | 0.21 | 0.21 |
| Yelkin TS³ | | | | | |
| Sucralose, | 0.10 | 0.00 | 0.00 | 0.00 | 0.00 |
| micronized | | | | | |
| Sucralose, | 0.00 | 0.93 | 0.00 | 0.00 | 0.00 |
| sample EU | | | | | |
| Sucralose, | 0.00 | 0.00 | 0.41 | 0.00 | 0.00 |
| sample I | | | | | |
| Sucralose, | 0.00 | 0.00 | 0.00 | 0.47 | 0.00 |
| sample R | | | | | |
| Sucralose, | 0.00 | 0.00 | 0.00 | 0.00 | 0.52 |
| sample S | | | | | |
| Sucralose, | 0.00 | 0.00 | 0.00 | 0.00 | 0.00 |
| sample T | | | | | |
| Sucralose, | 0.00 | 0.00 | 0.00 | 0.00 | 0.00 |
| sample U | | | | | |
| Sucralose, | 0.00 | 0.00 | 0.00 | 0.00 | 0.00 |
| sample V | | | | | |
| Sucralose, | 0.00 | 0.00 | 0.00 | 0.00 | 0.00 |
| sample W | | | | | |
| Sucralose, | 0.00 | 0.00 | 0.00 | 0.00 | 0.00 |
| sample X | | | | | |
| TOTAL | 100.00 | 100.00 | 100.00 | 100.00 | 100.00 |
| | Percent | | | | |
| Ingredient | Sample T | Sample U | Sample V | Sample W | Sample X |
| Crystalline | 54.86 | 54.86 | 54.86 | 54.45 | 54.45 |
| sorbitol, | | | | | |
| Sorbogem 712¹ | | | | | |
| Chewing gum | 29.74 | 29.74 | 29.74 | 29.74 | 29.74 |
| base, Eurodent | | | | | |
| T-RO² | | | | | |
| Maltitol syrup, | 12.31 | 12.31 | 12.31 | 12.31 | 12.31 |
| MALTISWEET 3145¹ | | | | | |
| Mannitol | 2.36 | 2.36 | 2.36 | 2.36 | 2.36 |
| powder¹ | | | | | |
| Soy lecithin, | 0.21 | 0.21 | 0.21 | 0.21 | 0.21 |
| Yelkin TS³ | | | | | |
| Sucralose, | 0.00 | 0.00 | 0.00 | 0.00 | 0.00 |
| micronized | | | | | |
| Sucralose, | 0.00 | 0.00 | 0.00 | 0.00 | 0.00 |
| sample EU | | | | | |
| Sucralose, | 0.00 | 0.00 | 0.00 | 0.00 | 0.00 |
| sample I | | | | | |
| Sucralose, | 0.00 | 0.00 | 0.00 | 0.00 | 0.00 |
| sample R | | | | | |
| Sucralose, | 0.00 | 0.00 | 0.00 | 0.00 | 0.00 |
| sample S | | | | | |
| Sucralose, | 0.53 | 0.00 | 0.00 | 0.00 | 0.00 |
| sample T | | | | | |
| Sucralose, | 0.00 | 0.53 | 0.00 | 0.00 | 0.00 |
| sample U | | | | | |
| Sucralose, | 0.00 | 0.00 | 0.53 | 0.00 | 0.00 |
| sample V | | | | | |
| Sucralose, | 0.00 | 0.00 | 0.00 | 0.93 | 0.00 |
| sample W | | | | | |
| Sucralose, | 0.00 | 0.00 | 0.00 | 0.00 | 0.93 |
| sample X | | | | | |
| TOTAL | 100.00 | 100.00 | 100.00 | 100.00 | 100.00 |

PROCEDURE:
1. Place pre-weighed gum base in a 45-50° C. (110-120° F.) for at least 30 minutes prior to use to soften the gum base.
2. To the pre-heated gum mixer (45-50° C.), add about half of the sorbitol powder and all of the mannitol power.
3. Start mixing.
4. Add the gum base and soy lecithin to the mixer.
5. Mix for about 1-2 minutes. Stop the mixer and make sure all of the gum base and other ingredients are incorporated.
6. Add the remaining sorbitol powder and maltitol syrup and start the mixer. Mix for 1-2 minutes and check again that all the ingredients are well incorporated.
7. Add the sucralose and any flavors to the mixer. Mix for 1-2 minutes or until smooth and well-incorporated.
8. Laminate the gum to 3 mm thickness (mannitol may be spread on the surface of the gum to help with stickiness, if needed).
9. Cut the gum into 3.0 ± 0.05 gram pieces. Wrap in wax paper or foil (be sure that the gum has cooled to room temperature before wrapping). Place wrapped pieces in foil bag and seal.
10. Allow the gum to rest for at least 24 hours before evaluating.
¹Corn Products, Specialty Ingredients division
²Eurobase
³ADM

### 7.5 Sensory Evaluation.

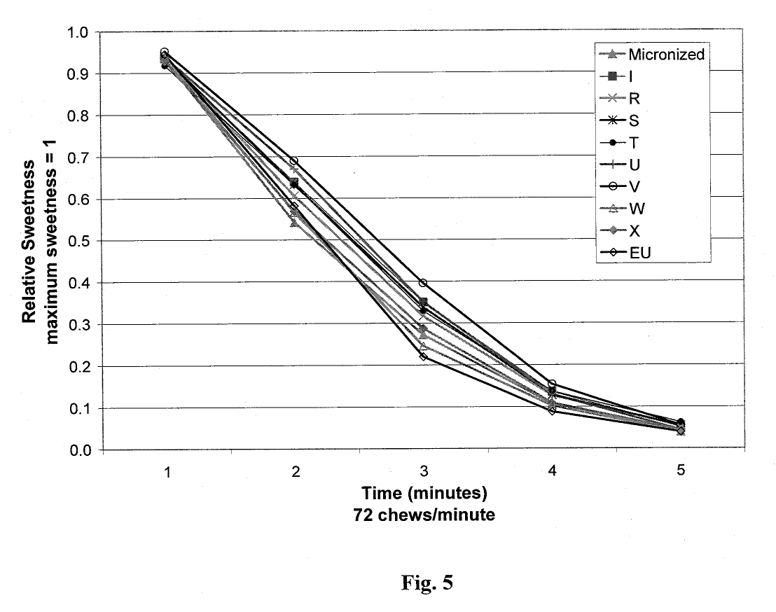

Ten panelists were instructed in magnitude estimation with sucrose solutions, in which panelists estimated the magnitude of increase or decrease in a sensory attribute relative to a reference. In separate tests, panelists were instructed to note the maximum sweetness of chewing gum samples, usually between 20 and 30 seconds of chewing, and to estimate the amount of sweetness remaining at 1 through 5 minutes (at each minute interval), relative to the maximum sweetness. Chewing rate was controlled with a metronome at 72 beats per minute. The panelists were served a 3.0±0.05 gram piece of gum wrapped in waxed paper in a soufflé cup labeled with a random three-digit code. The products were evaluated in a randomized order in triplicate over 6 days. Each panelist had their own presentation order. Each panelist evaluated 5 samples per day. Evaluation sessions were one hour per day. Bottled water and unsalted crackers were available before the testing and between samples for the panelists to clear their palates. The sweetness profiles obtained by these methods are given in Table 27 and FIG. 5.

TABLE 27
Sweetness Relative To Maximum Sweetness

| Sucralose | 1 | 2 | 3 | 4 | 5 |
|---|---|---|---|---|---|
| Sample | minute | minutes | minutes | minutes | minutes |
| V | 0.95 | 0.69 | 0.40 | 0.15 | 0.06 |
| U | 0.94 | 0.67 | 0.35 | 0.13 | 0.04 |
| S | 0.94 | 0.63 | 0.34 | 0.13 | 0.05 |
| I | 0.93 | 0.64 | 0.35 | 0.14 | 0.05 |
| T | 0.92 | 0.63 | 0.33 | 0.14 | 0.06 |
| R | 0.94 | 0.61 | 0.32 | 0.13 | 0.06 |
| X | 0.93 | 0.57 | 0.29 | 0.11 | 0.04 |
| Micronized | 0.93 | 0.54 | 0.27 | 0.11 | 0.04 |
| W | 0.94 | 0.57 | 0.25 | 0.10 | 0.04 |
| EU | 0.94 | 0.58 | 0.22 | 0.09 | 0.04 |

* No significant differences between sucralose samples at each time interval

No significant difference in relative sweetness between chewing samples made with different sucralose samples existed at each minute of chewing. Sample V, sucralose absorbed into bentonite and coated with gum arabic, maintained a higher level of sweetness through 5 minutes. Gum Arabic consists of a low molecular weight polysaccharide and a high molecular weight hydroxyproline-rich glycoprotein. The combination of the hydrophilic carbohydrate and hydrophobic protein enables the sample coated with gum Arabic to interact with the hydrophobic nature of chewing gum base and the hydrophilic nature of sucralose and bentonite. In general, a hydrocolloid coating further extended the sweetness release from chewing gum compared to gums containing samples that did not have hydrocolloid coatings.

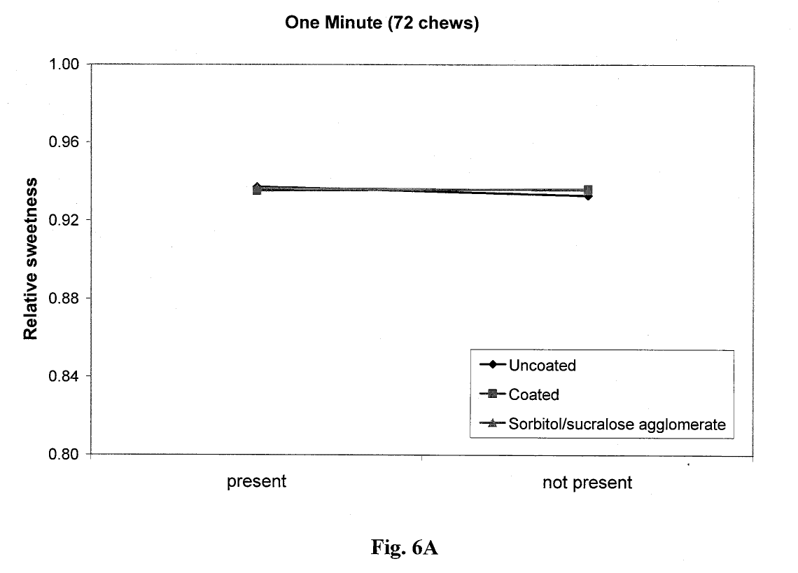

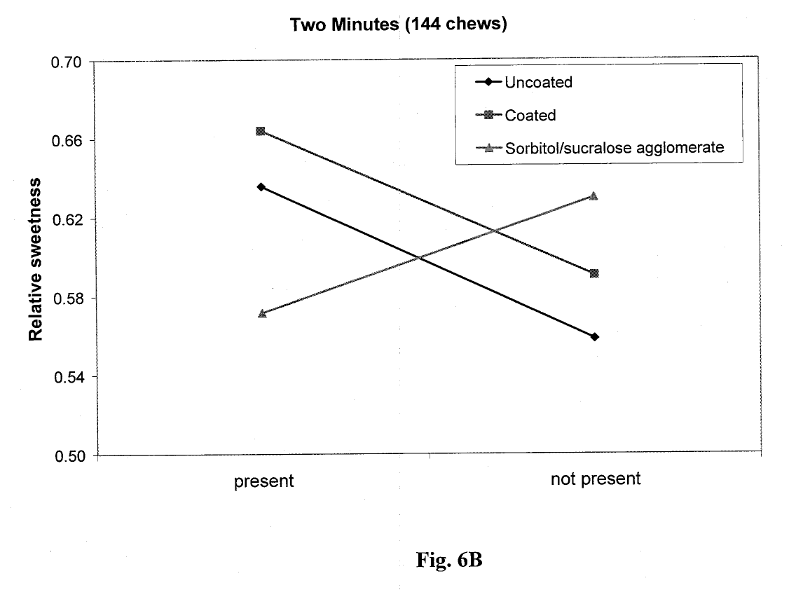

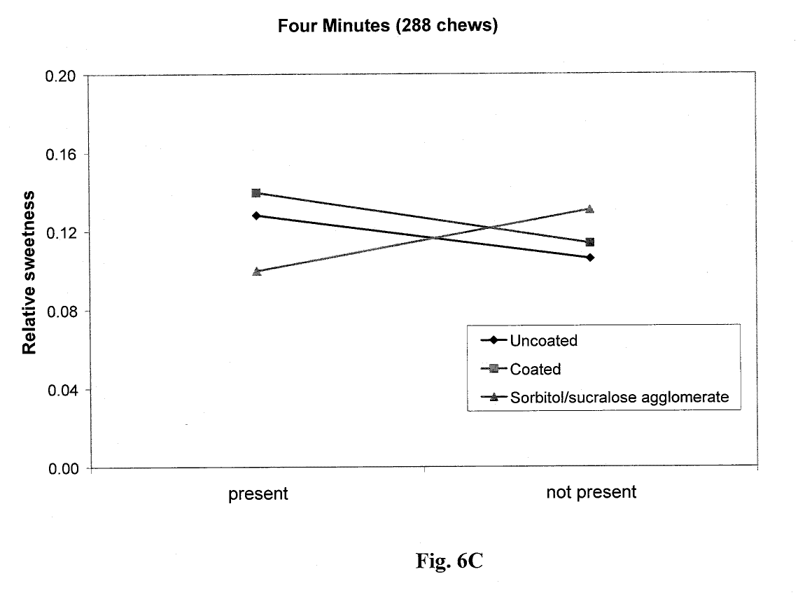

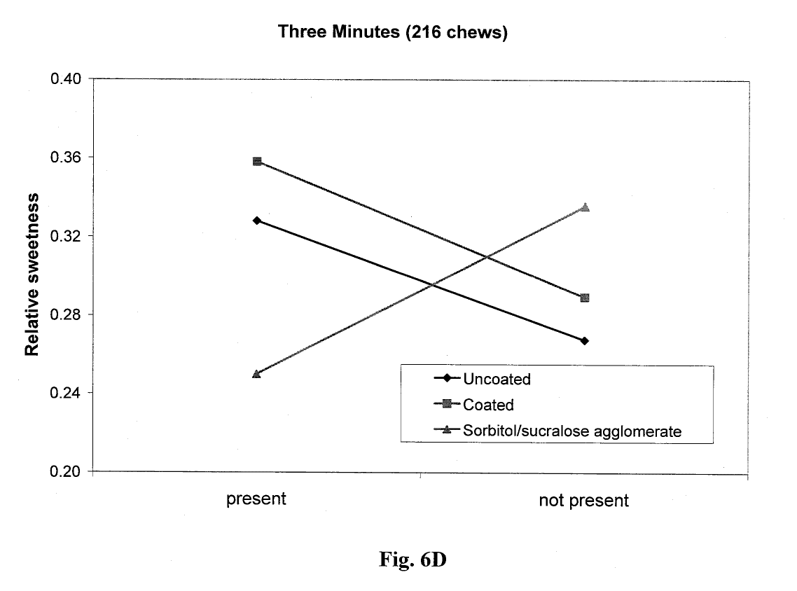

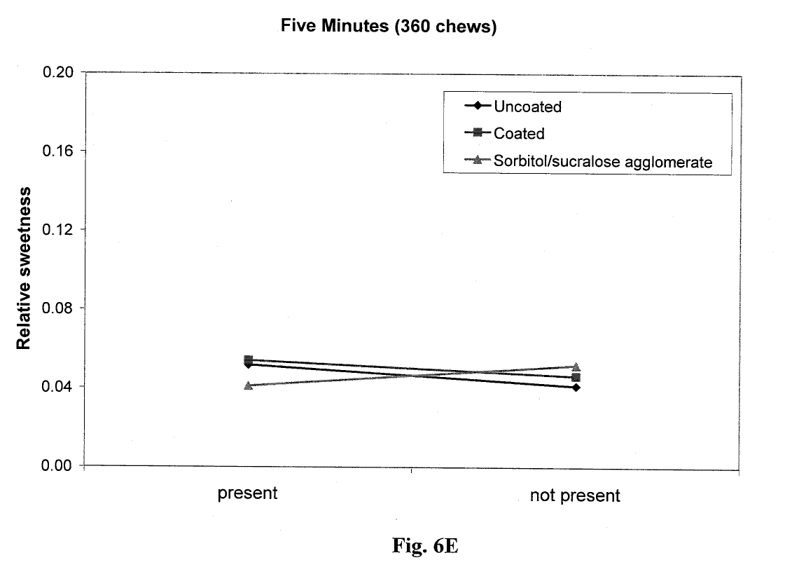

Coatings of guar gum or iota-carrageenan offered less benefit than gum arabic in extending the sweetness compared with uncoated bentonite with sucralose. To further understand the effect of a hydrocolloid coating on bentonite/sucralose samples, the interaction between coated and uncoated can be compared at each minute of chewing (FIGS. 6A-E, representing minutes 1-5, respectively).

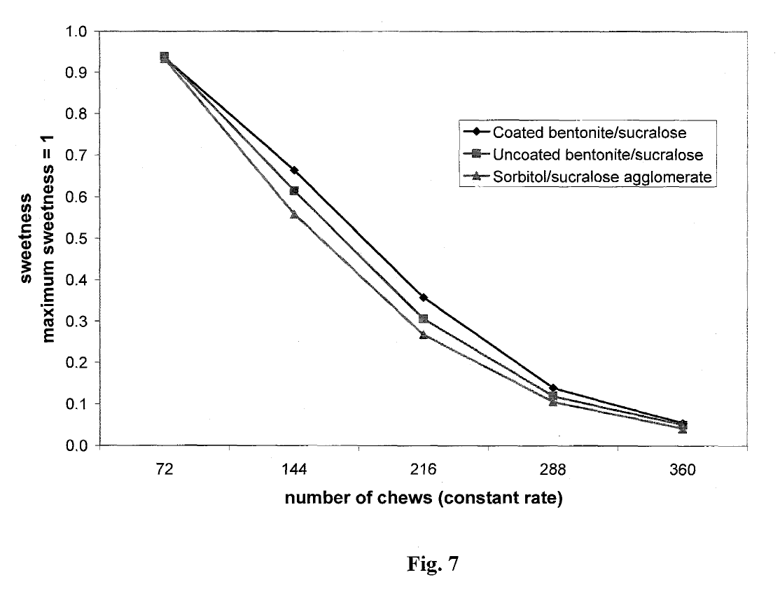

At one minute and five minutes of chewing, the sucralose form has little effect on the sweetness of chewing gum. At two, three and four minutes, the presence of bentonite/sucralose samples tended to increase the sweetness relative to non-bentonite samples. The presence of a coating on the bentonite samples had the same relative effect as the uncoated bentonite samples, but the coating helped to increase the relative sweetness at each minute of chew. This result is summarized in FIG. 7.

Three different samples of granulated sucralose absorbed into bentonite were made and tested in chewing gum. The samples contained the same sucralose content and were processed under similar conditions, except that the particle size distribution of the final ingredient varies (FIG. 8).

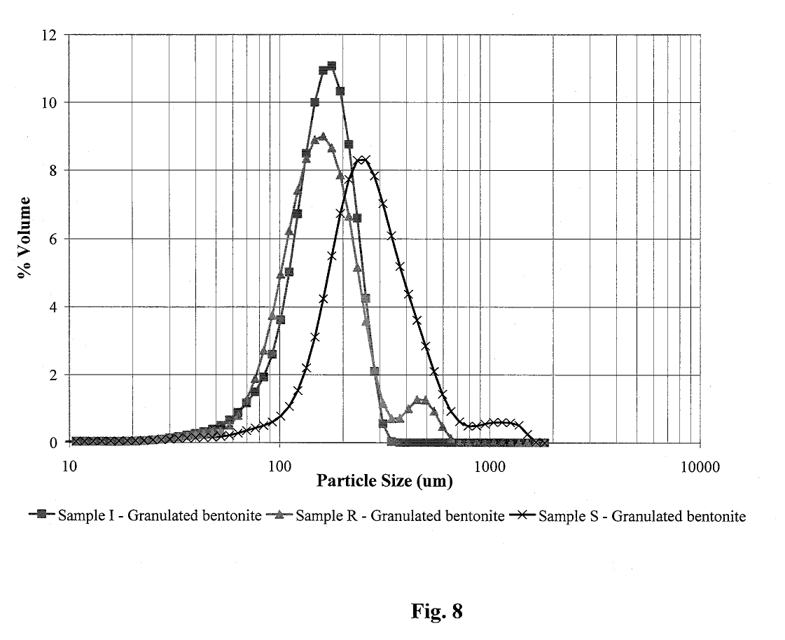

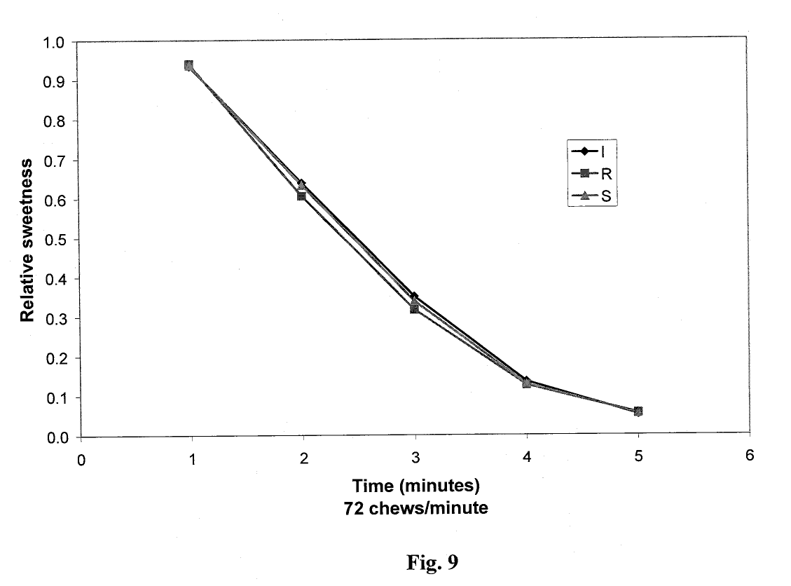

Differences in particle size of the granulated bentonite with sucralose samples did not translate to differences in the sweetness perception in chewing gum (FIG. 9).

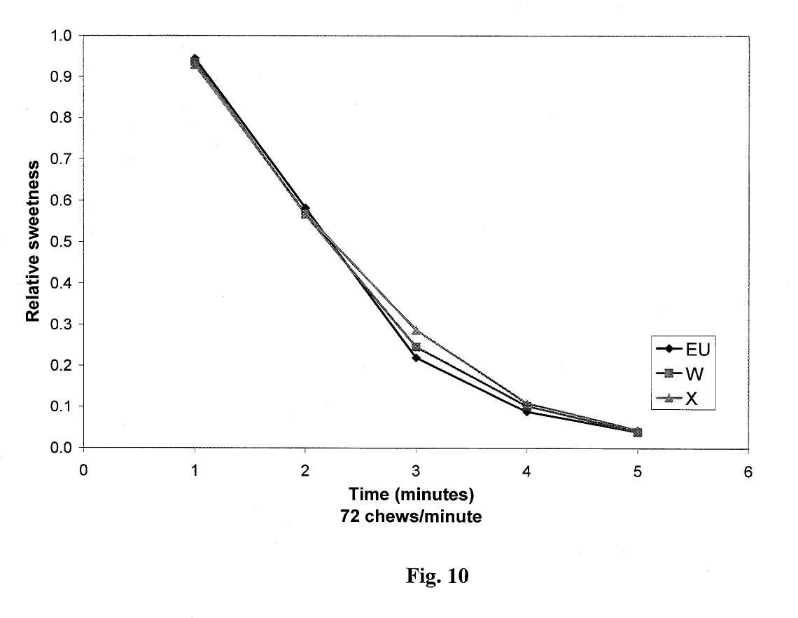

Similarly, for the sorbitol/sucralose agglomerate, particle size did not seem to have an effect on the sweetness profile in chewing gum (FIG. 10).

Particle size does not have an effect on sweetness release in chewing gum within the range tested. Granulated bentonite with sucralose maintained the same sweetness profile in chewing gum at particle sizes of 169, 184 and 324 μm. Sorbitol/sucralose agglomerates maintained the same sweetness profile in chewing gum at particle sizes of 324, 354, and 722 μm.

### Example 8

### Effect of Sucralose Concentration on Sweetness and Maximum Sweetness Perception

The goal the following experiments was to identify the effect of the concentration of sucralose in the bentonite carrier and the effect of the total sucralose concentration in the chewing gum on its sweetness release and maximum sweetness perception.

### 8.1 Materials.

| | |
|---|---|
| Bentonite | BPM Minerals; National Premium WT 325 |
| Sucralose | Tate & Lyle; DFF-1 |
| Sorbitol | Corn Products; Sorbogem 712 |

### 8.2 Sustained Release Sample Preparation.

Granulation was carried out on the Glatt ProCell 5 with the bottom-spray configuration on the AGT insert. An aqueous mixture of 10% bentonite and 11.25 or 22.5% sucralose was hydrated in a sonic bath for 1 hour and used as the spray. The bed consisted of 18 micron bentonite particles

Coating was carried out on the Glatt ProCell 5 with the bottom-spray configuration on the AGT insert. The bed consisted of material from the granulation process, with a spray of 0.5% gum arabic solution.

Product compositions are summarized in Table 28.

TABLE 28
Experimental Sucralose Samples

| | | Spray | | | Final |
|---|---|---|---|---|---|
| Sample | Conditions | fraction | | (g) | Fraction |
| F4-884, 8% | 11.25% | | Spray | 431 | |
| sucralose | sucralose | | | | |
| 7145-125 | GF insert, | | Bed | 500 | |
| | bottom-spray | | | | |
| | Bentonite bed | 0.1 | Bentonite | 43.10 | 0.92 |
| | | 0.1125 | Sucralose | 48.49 | 0.08 |
| | | | Dry total | 591.59 | 1.00 |
| F4-885, | 11.25% | | Spray | 400 | |
| 10% | sucralose | | | | |
| sucralose | | | | | |
| 7145-127 | GF insert, | | Bed | 200 | |
| | bottom-spray | | | | |
| | F4-884, 8% | | Bentonite | 178.89 | 0.88 |
| | sucralose bed | | | | |
| | | | Sucralose | 21.11 | 0.10 |
| | | 0.01 | Gum | 4.00 | 0.02 |
| | | | arabic | | |
| | | | Dry total | 204.00 | 1.00 |
| F4-884, | 22.5% sucralose | | Spray | 455 | |
| 16% | | | | | |
| sucralose | | | | | |
| 7145-126 | GF insert, | | Bed | 500 | |
| | bottom-spray | | | | |
| | Bentonite bed | 0.1 | Bentonite | 45.50 | 0.84 |
| | | 0.225 | Sucralose | 102.38 | 0.16 |
| | | | Dry total | 647.88 | 1.00 |
| F4-885, | 22.5% sucralose | | Spray | 319 | |
| 16% | | | | | |
| sucralose | | | | | |
| 7145-127 | GF insert, | | Bed | 200 | |
| | bottom-spray | | | | |
| | F4-884, 16% | | Bentonite | 168.40 | 0.83 |
| | sucralose bed | | | | |
| | | | Sucralose | 31.60 | 0.16 |
| | | 0.01 | Gum | 3.19 | 0.02 |
| | | | arabic | | |
| | | | Dry total | 203.19 | 1.00 |

Particle size distribution of each experimental sucralose sample listed in Table 28 was analyzed on the Beckman LS 13 320 Laser Diffraction Particle Size Analyzer using the tornado powder system with the Standard Operating Procedure (SOP) for “dry powder” (FIG. 11 and Table 29).

TABLE 29
Mean Particle Size of the Experimental Sucralose Samples

| | | | Mean |
|---|---|---|---|
| Sucralose | | % | Particle |
| Sample | Description | Sucralose | Size (μm) |
| Micronized | Micronized sucralose | 100.00 | 5.31 |
| F4-884, 8% | 11.25% sucralose absorbed into | 8.00 | 146.37 |
| sucralose | bentonite, granulated on top of a | | |
| | bentonite seed | | |
| F4-885, 10% | F4-884, 8% sucralose coated with | 0.10 | 171.86 |
| sucralose | gum arabic¹ | | |
| F4-884, 16% | 22.5% sucralose absorbed into | 16.00 | 262.67 |
| sucralose | bentonite, granulated on top of a | | |
| | bentonite seed | | |
| F4-885, 16% | F4-885, 16% sucralose coated with | 16.00 | 288.05 |
| sucralose | gum arabic¹ | | |

¹FIBERGUM from Colloides Naturels International

### 8.3 Chewing Gum Preparation.

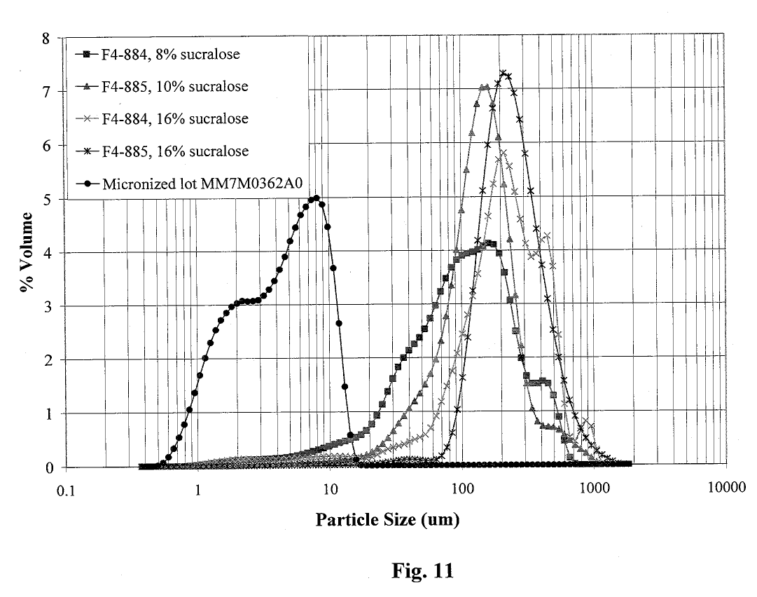

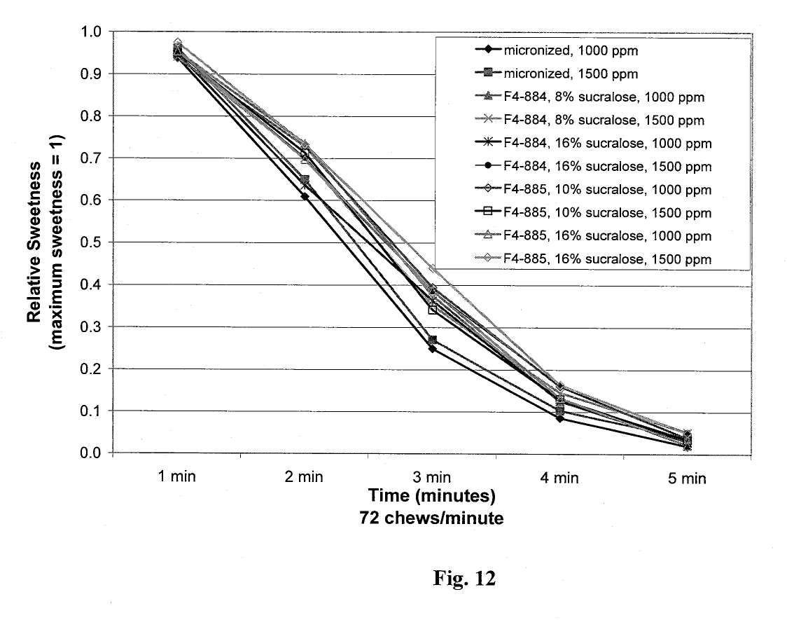

Ten batches of chewing gum were prepared, five containing 1000 ppm sucralose and five containing 1500 ppm sucralose, based on the theoretical sucralose concentration for the sucralose ingredients described in Table 30 and FIG. 12. Chewing gum was prepared in 1500 gram batch sizes using a Littleford twin sigma blade gum mixer at the University of Wisconsin-Madison. Formulas are given in Table 31.

TABLE 30
Sweetness Relative To Maximum Sweetness

| | 1 | | | 4 | 5 |
|---|---|---|---|---|---|
| Sucralose Sample | minute | 2 minutes | 3 minutes | minutes | minutes |
| F4-885, 16% sucralose, 1500 ppm | 0.972^{a} | 0.730^{a} | 0.435^{a} | 0.161^{a} | 0.050^{ab} |
| Micronized, 1500 ppm | 0.956^{a} | 0.634^{ab} | 0.251^{ab} | 0.094^{a} | 0.039^{ab} |
| F4-885, 16% sucralose, 1000 ppm | 0.950^{a} | 0.684^{ab} | 0.369^{ab} | 0.129^{a} | 0.023^{b} |
| F4-884, 16% sucralose, 1500 ppm | 0.950^{a} | 0.698^{ab} | 0.381^{ab} | 0.164^{a} | 0.054^{ab} |
| F4-884, 8% sucralose, 1000 ppm | 0.949^{a} | 0.732^{ab} | 0.364^{ab} | 0.121^{a} | 0.037^{ab} |
| F4-885, 10% sucralose, 1500 ppm | 0.948^{a} | 0.710^{ab} | 0.337^{b} | 0.134^{a} | 0.035^{a} |
| F4-884, 16% sucralose, 1000 ppm | 0.946^{a} | 0.632^{b} | 0.362^{ab} | 0.126^{a} | 0.031^{ab} |
| F4-884, 8% sucralose, 1500 ppm | 0.935^{a} | 0.696^{ab} | 0.335^{b} | 0.137^{a} | 0.053^{ab} |
| F4-885, 10% sucralose, 1000 ppm | 0.934^{a} | 0.723^{a} | 0.386^{ab} | 0.160^{a} | 0.041^{ab} |
| Micronized, 1000 ppm | 0.930^{a} | 0.590^{ab} | 0.236^{ab} | 0.081^{a} | 0.019^{ab} |

^{ab}means in a column followed by the same letter are not significantly different at α = 0.05

TABLE 31
Formulas

| | Percent | | | | |
|---|---|---|---|---|---|
| | Micronized, | Micronized, | F4-884, 8% | F4-884, 8% | F4-885, 10% |
| Ingredient | 1000 ppm | 1500 ppm | sucralose, 1000 ppm | sucralose, 1500 ppm | sucralose, 1000 ppm |
| Crystalline | 55.28 | 55.23 | 54.10 | 53.46 | 54.36 |
| sorbitol, | | | | | |
| Sorbogem 712¹ | | | | | |
| Chewing gum | 29.74 | 29.74 | 29.74 | 29.74 | 29.74 |
| base, Eurodent | | | | | |
| T-RO² | | | | | |
| Maltitol syrup, | 12.31 | 12.31 | 12.31 | 12.31 | 12.31 |
| MALTISWEET | | | | | |
| 3145¹ | | | | | |
| Mannitol | 2.36 | 2.36 | 2.36 | 2.36 | 2.36 |
| powder | | | | | |
| Soy lecithin, | 0.21 | 0.21 | 0.21 | 0.21 | 0.21 |
| Yelkin TS³ | | | | | |
| Sucralose, | 0.10 | 0.15 | 0.00 | 0.00 | 0.00 |
| micronized | | | | | |
| F4-884, 8% | 0.00 | 0.00 | 1.28 | 1.92 | 0.00 |
| sucralose | | | | | |
| F4-885, 10% | 0.00 | 0.00 | 0.00 | 0.00 | 1.02 |
| sucralose | | | | | |
| F4-884, 16% | 0.00 | 0.00 | 0.00 | 0.00 | 0.00 |
| sucralose | | | | | |
| F4-885, 16% | 0.00 | 0.00 | 0.00 | 0.00 | 0.00 |
| sucralose | | | | | |
| TOTAL | 100.00 | 100.00 | 100.00 | 100.00 | 100.00 |

| | Percent | | | | |
|---|---|---|---|---|---|
| | | | | F4-885, 16% | |
| | F4-885, 10% | F4-884, 16% | F4-884, 16% | sucralose, | F4-885, 16% |
| Ingredient | sucralose, 1500 ppm | sucralose, 1000 ppm | sucralose, 1500 ppm | 1000 ppm | sucralose, 1500 ppm |
| Crystalline | 53.85 | 54.74 | 54.42 | 54.74 | 54.42 |
| sorbitol, | | | | | |
| Sorbogem 712¹ | | | | | |
| Chewing gum | 29.74 | 29.74 | 29.74 | 29.74 | 29.74 |
| base, Eurodent | | | | | |
| T-RO² | | | | | |
| Maltitol syrup, | 12.31 | 12.31 | 12.31 | 12.31 | 12.31 |
| MALTISWEET | | | | | |
| 3145¹ | | | | | |
| Mannitol powder | 2.36 | 2.36 | 2.36 | 2.36 | 2.36 |
| Soy lecithin, | 0.21 | 0.21 | 0.21 | 0.21 | 0.21 |
| Yelkin TS³ | | | | | |
| Sucralose, | 0.00 | 0.00 | 0.00 | 0.00 | 0.00 |
| micronized | | | | | |
| F4-884, 8% | 0.00 | 0.00 | 0.00 | 0.00 | 0.00 |
| sucralose | | | | | |
| F4-885, 10% | 1.53 | 0.00 | 0.00 | 0.00 | 0.00 |
| sucralose | | | | | |
| F4-884, 16% | 0.00 | 0.64 | 0.96 | 0.00 | 0.00 |
| sucralose | | | | | |
| F4-885, 16% | 0.00 | 0.00 | 0.00 | 0.64 | 0.96 |
| sucralose | | | | | |
| TOTAL | 100.00 | 100.00 | 100.00 | 100.00 | 100.00 |

¹Corn Products, Specialty Ingredients division
²Eurobase
³ADM

### Procedure:

1. Place pre-weighed gum base in a 45-50° C. (110-120° F.) for at least 30 minutes prior to use to soften the gum base.
2. To the pre-heated gum mixer (45-50° C.), add about half of the sorbitol powder and all of the mannitol power.
3. Start mixing.
4. Add the gum base and soy lecithin to the mixer.
5. Mix for about 1-2 minutes Stop the mixer and make sure all of the gum base and other ingredients are incorporated.
6. Add the remaining sorbitol powder and maltitol syrup and start the mixer. Mix for 1-2 minutes and check again that all the ingredients are well incorporated.
7. Add the sucralose and any flavors to the mixer. Mix for 1-2 minutes or until smooth and well-incorporated.
8. Laminate the gum to 3 mm thickness (mannitol may be spread on the surface of the gum to help with stickiness, if needed).
9. Cut the gum into 3.0±0.05 gram pieces. Wrap in wax paper or foil (be sure that the gum has cooled to room temperature before wrapping). Place wrapped pieces in foil bag and seal.
10. Allow the gum to rest for at least 24 hours before evaluating.

### 8.4 Relative Sweetness Profile.

Nine panelists were instructed in magnitude estimation with sucrose solutions. Using this method, panelists estimated the magnitude of increase or decrease in a sensory attribute relative to a reference. In separate tests, panelists were instructed to estimate the amount of sweetness remaining over the course of 5 minutes (at each minute interval), relative to the maximum sweetness (usually perceived between 20 and 60 seconds of chewing). Chewing rate was controlled with a metronome at 72 beats per minute. The panelists were served a 3.0±0.05 gram piece of gum wrapped in parchment paper in a soufflé cup labeled with a random three-digit code. The products were evaluated in a randomized order in triplicate over 6 days. Each panelist had their own presentation order. Each panelist evaluated 5 samples per day. Evaluation sessions were one hour per day. Bottled water and unsalted crackers were available before the testing and between samples for the panelists to clear their palates.

### 8.5 Maximum Sweetness.

Nine semi-trained panelists were instructed to note the maximum sweetness of chewing gum samples, usually between 20 and 60 seconds of chewing, relative to reference sucrose solutions of 10, 15 and 20% sucrose. Chewing rate was controlled with a metronome at 72 beats per minute. The panelists were served a 3.0±0.05 gram piece of gum wrapped in parchment paper in a soufflé cup labeled with a random three-digit code. The products were evaluated in a randomized order in triplicate over 3 days. Each panelist had their own presentation order. Bottled water and unsalted crackers were available before the testing and between samples for the panelists to clear their palates.

### 8.6 Overall Effects.

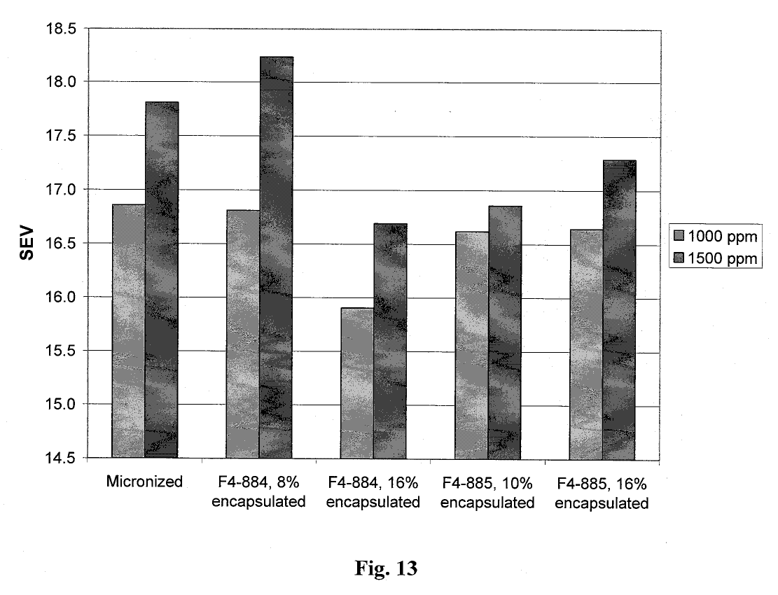

Overall, significant differences between the sucralose samples in the chewing gum were minimal. No significant differences existed in the relative sweetness of the chewing gum at 1 minute and 4 minutes of chewing (Table 32, FIG. 13). In general, micronized sucralose, when incorporated at 1000 and 1500 ppm in chewing gum, had the fastest sweetness release, while chewing gum containing F4-885 with 16% encapsulated sucralose at 1500 ppm sucralose in the gum had the slowest release.

Panelists had difficulty in determining the maximum sweetness of the chewing gum. Differences between sucralose samples were minimal and may have little meaning in a practical sense. The only significant difference in maximum sweetness between the chewing gums was between chewing gum containing F4-884 with 8% encapsulated sucralose with 1500 ppm sucralose in the chewing gum and F4-884 with 16% encapsulated sucralose with 1000 ppm sucralose in the gum.

TABLE 32
Chewing Gum Maximum Sweetness

| | Maximum |
|---|---|
| | Sweetness |
| Sucralose Sample | (SEV) |
| F4-884, 8% sucralose, 1500 ppm | 18.24^{a} |
| Micronized, 1500 ppm | 17.81^{ab} |
| F4-885, 16% sucralose, 1500 ppm | 17.29^{ab} |
| Micronized, 1000 ppm | 16.86^{ab} |
| F4-885, 10% sucralose, 1500 ppm | 16.86^{ab} |
| F4-884, 8% sucralose, 1000 ppm | 16.81^{ab} |
| F4-884, 16% sucralose, 1500 ppm | 16.69^{ab} |
| F4-885, 16% sucralose, 1000 ppm | 16.64^{ab} |
| F4-885, 10% sucralose, 1000 ppm | 16.62^{ab} |
| F4-884, 16% sucralose, 1000 ppm | 15.90^{b} |

^{ab}means in a column followed by the same letter are not significantly different at α = 0.05

### 8.7 Effect of Sucralose Level in the Chewing Gum.

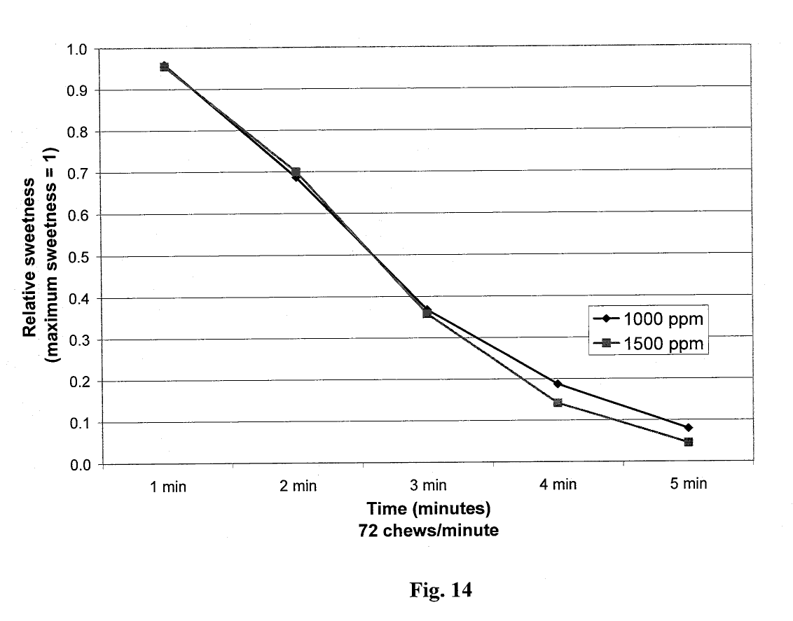

The level of sucralose in the chewing gum, 1000 ppm or 1500 ppm, did not have much effect on the relative sweetness release, particularly at 1, 2 and 3 minutes of chewing. After 4 and 5 minutes of chew, gum containing 1000 ppm of sucralose retained more of its sweetness than gum containing 1500 ppm sucralose (FIG. 14).

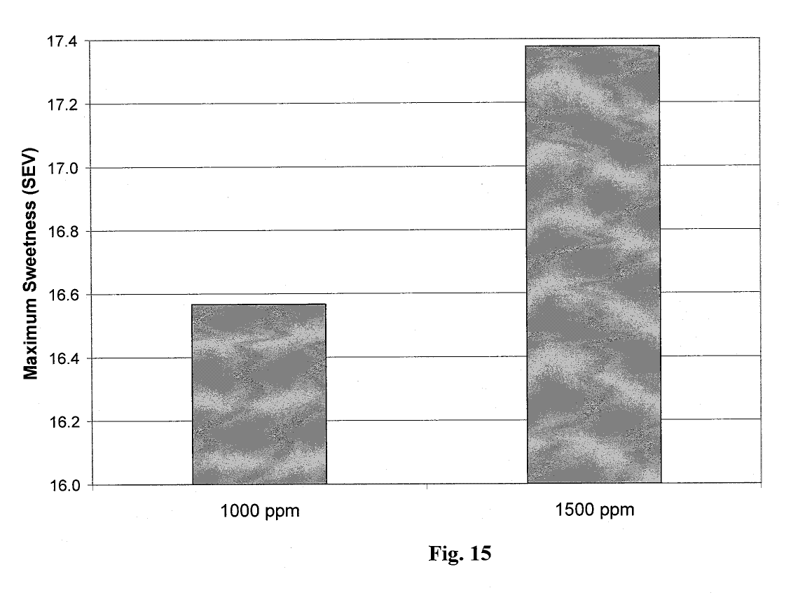

As might be expected, chewing gum with 1500 ppm of sucralose had a higher maximum sweetness (17.38 SEV) than gum with 1000 ppm (16.57 SEV) (FIG. 15).

### 8.8 Effect of Sucralose Form.

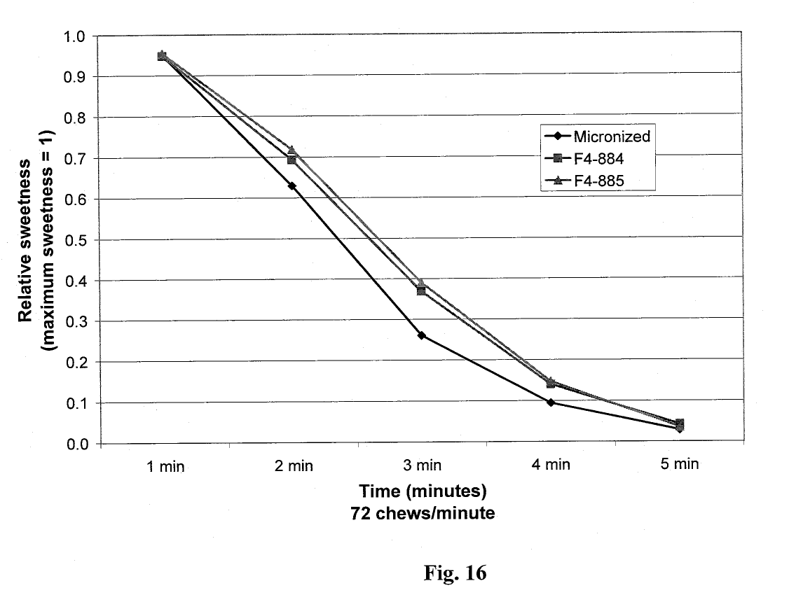

Encapsulating sucralose in bentonite had a considerable effect on extending the sweetness release in chewing gum compared to micronized sucralose. The effect of the gum arabic coating on the bentonite/sucralose product was less pronounced and may only have minor, if any, practical implications (FIG. 16). Example 7 shows that chewing gum products with hydrocolloid coatings showed enhanced sweetness retention over the control samples with and without bentonite (micronized sucralose and a granulated sucralose bentonite product). Of the hydrocolloids tested, gum arabic showed the most promise.

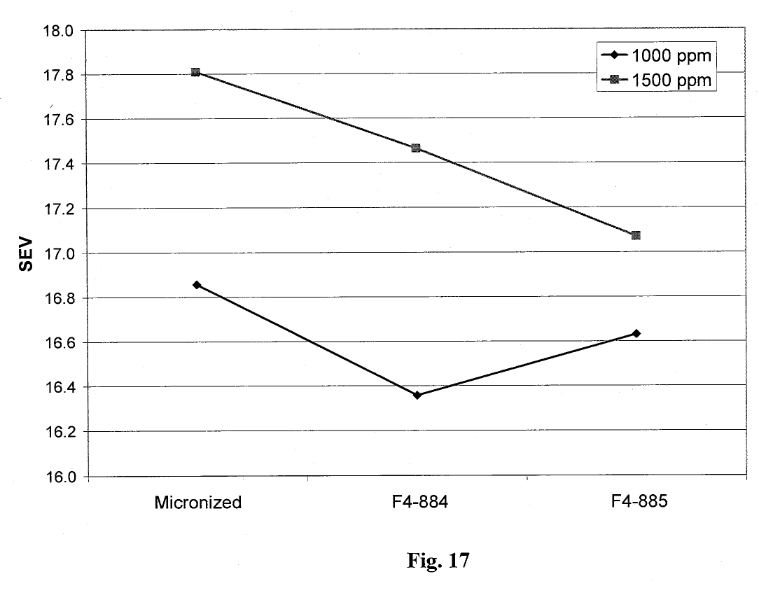

The sucralose/bentonite products also had lower maximum sweetness scores compared to the chewing gum made with micronized sucralose (FIG. 17). Possibly, some of the sucralose in the sucralose/bentonite products (F4-884 and F4-885) is retained in the gum in the early stages of chewing so that the initial sweetness impact is reduced, and over time, that sucralose is released, which results in the extended sweetness profile see in FIG. 16. Alternatively, some of the sucralose may be entrapped in the gum base, preventing its release altogether, which may also account for the lower maximum sweetness of the F4-884 and F4-885 chewing gums compared to the micronized sucralose gum.

When incorporated at 1000 ppm sucralose, the difference in the maximum SEV between F4-884 and F4-885 was small. At 1500 ppm, F4-885 lowered the maximum sweetness by approximately 0.5 SEV, which may be due to the delayed sweetness release or the prohibition of release altogether (FIG. 17).

### 8.9 Effect of Sucralose Encapsulation Level.

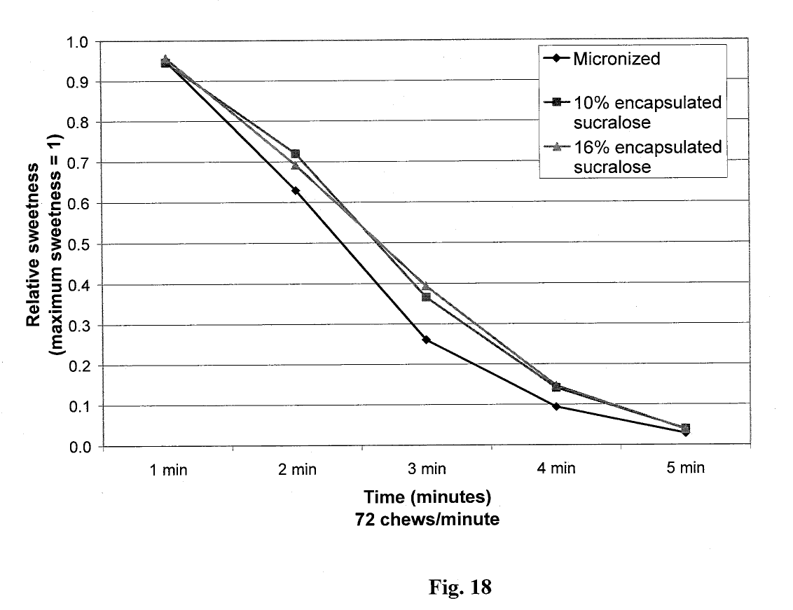

As reported above and seen in FIG. 18, encapsulating sucralose in bentonite extended the sweetness release in chewing gum compared to micronized sucralose. However, the amount of sucralose encapsulated, 8-10% or 16%, had negligible and inconsistent effects on the sweetness release.

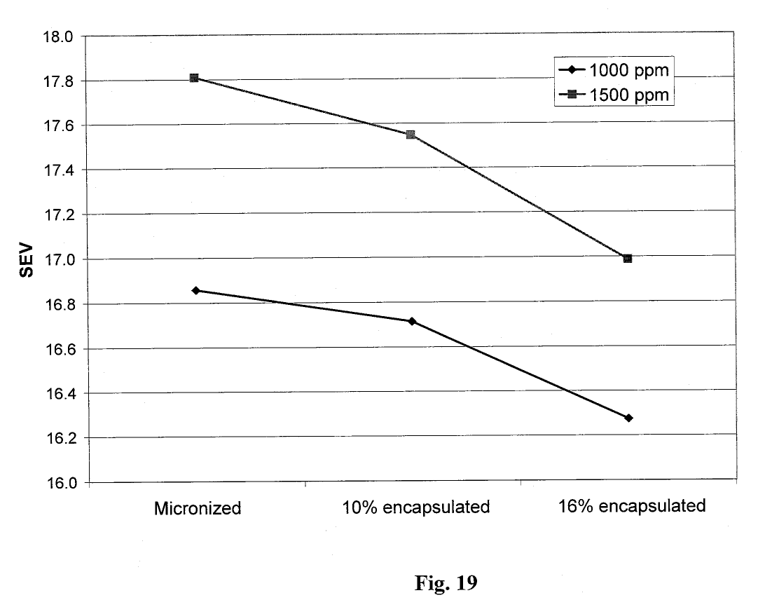

Increasing the percentage of sucralose in the sucralose/bentonite product decreased the maximum sweetness perception at both 1000 and 1500 ppm sucralose in the gum (FIG. 19). This may be due to delayed sweetness release or to prohibition of sucralose release.

The level of sucralose in the chewing gum, 1000 ppm or 1500 ppm, did not have much effect on the relative sweetness release, particularly at 1, 2 and 3 minutes of chewing. After 4 and 5 minutes of chew, gum containing 1000 ppm of sucralose retained more of its sweetness than gum containing 1500 ppm sucralose. As might be expected, chewing gum with 1500 ppm of sucralose had a higher maximum sweetness (17.38 SEV) than gum with 1000 ppm (16.57 SEV).

Encapsulating sucralose in bentonite had a considerable effect on extending the sweetness release in chewing gum compared to micronized sucralose. The effect of the gum arabic coating on the bentonite/sucralose product was less pronounced and may only have minor, if any, practical implications. The sucralose/bentonite products also had lower maximum sweetness scores compared to the chewing gum made with micronized sucralose. This phenomenon may be due to the delayed sweetness release or the prohibition of release altogether.

The amount of sucralose encapsulated, 8-10% or 16%, had negligible and inconsistent effects on the rate of sweetness release. Increasing the percentage of sucralose in the sucralose/bentonite product decreased the maximum sweetness perception at both 1000 and 1500 ppm sucralose in the gum.

1. A co-agglomerate of sucralose and a polyol, wherein the co-agglomerate comprises grains of polyol onto which the sucralose has been dried.

2. The co-agglomerate of claim 1, wherein the sucralose forms at least a partial coating or particles on the grain of the polyol.

3. The co-agglomerate of claim 1, wherein the polyol is sorbitol, maltitol, or a mixture thereof.

4. The co-agglomerate of claim 1, wherein the co-agglomerate has a sucralose-polyol ratio from about 0.05 wt.-% to about 40 wt-%.

5. The co-agglomerate of claim 4, wherein the co-agglomerate has a sucralose-polyol ratio from about 0.15 wt.-% to about 2.20 wt.-%, about 0.20 wt.-% to about 0.70 wt.-%, or about 0.25 wt.-% to about 0.40 wt.-%.

6. The co-agglomerate of claim 4 wherein the co-agglomerate has a sucralose-polyol ratio from about 5 wt.-% to about 40 wt.-% or about 10 wt.-% to about 25 wt.-%.

7. A food composition comprising the co-agglomerate of claim 1.

8. The food composition of claim 7, wherein the food composition is chewing gum.

9. The food composition of claim 7, wherein the food composition is a chewing gum comprising one or more ingredients selected from the group consisting of chewable gum base, flavor, bulking agent, and softener.

10. The food composition of claim 9, wherein the flavor comprises mint.

11. A method for making the co-agglomerate of claim 1, comprising:
contacting a dry powder of polyol with a liquid solution of sucralose in a suitable solvent under conditions which moisten, but not dissolve the polyol powder, to form a moist mixture;
drying the moist mixture to form a co-agglomerate comprising grains of agglomerated polyol onto which sucralose has been dried.

12. The method of claim 11, wherein the polyol powder is pretreated by agglomeration or extrusion.

13. A pharmaceutical or cosmetic composition comprising the co-agglomerate of claim 1.

14. The food composition of claim 8, further comprising sucralose absorbed into and granulated on top of bentonite or sucralose encapsulated in bentonite.

15. The food composition of claim 14, wherein the sucralose is absorbed into and is granulated on top of bentonite.

16. The food composition of claim 14, wherein the sucralose is encapsulated in bentonite.

17. The food composition of claim 14, further comprising a guar gum coating.

18. The food composition of claim 14, further comprising an iota carrageenan coating.

19. The food composition of claim 14, further comprising a gum arabic coating.

20. A chewing gum comprising a first component for rapid flavor release and a second component for extended flavor release, the first component comprising a co-agglomerate comprising grains of polyol onto which sucralose has been dried, and the second component comprising bentonite into which sucralose has been absorbed and on top of which sucralose has been granulated.

21. The chewing gum of claim 20, wherein the polyol is sorbitol, maltitol, or a mixture thereof.

22. The chewing gum of claim 20, wherein the second component further comprises a gum arabic coating.

23. The chewing gum of claim 20, wherein a weight ratio of the first component and the second component is in a range from 1:9 to 9:1.
